(19) United States
(12) Patent Application Publication
(10) Pub. No.: US 2024/0076284 A1
(43) Pub. Date: Mar. 7, 2024

(54) NEW COMPOUNDS AND METHODS

(71) Applicant: BENEVOLENTAI BIO LIMITED, London (GB)

(72) Inventors: Rebecca PAUL, London (GB); Michael John RAWLING, London (GB); Christine WATSON, London (GB)

(73) Assignee: BENEVOLENTAI BIO LIMITED, London (GB)

(21) Appl. No.: 18/258,026

(22) PCT Filed: Dec. 16, 2021
(86) PCT No.: PCT/GB2021/053321

§ 371 Date: Jun. 16, 2023

(30) Foreign Application Priority Data
Dec. 16, 2020 (GB) 2019877.6

(51) Int. Cl. C07D 401/14

(52) U.S. Cl. CPC C07D 401/14

(57) ABSTRACT

The present invention relates to compounds of Formula (I) that are inhibitors of c-ABL. The invention also relates to pharmaceutical compositions comprising those compounds, and to their use in the treatment or prevention of medical conditions in which inhibition of c-ABL is beneficial. Such medical conditions include neurodegenerative diseases and cancer.

### FIELD OF THE INVENTION

The present invention relates to compounds of Formula (I) that are inhibitors of c-ABL. The invention also relates to pharmaceutical compositions comprising those compounds, and to their use in the treatment or prevention of medical conditions in which inhibition of c-ABL is beneficial. Such medical conditions include neurodegenerative diseases and cancer.

### BACKGROUND

ABL1 (Abelson Murine Leukaemia Viral Oncogene Homolog 1) is a protein that exhibits tyrosine kinase enzymatic activity and is associated with various cell functions. In humans, this protein is encoded by the ABL1 gene located on chromosome 9. The version of the ABL1 gene found within the mammalian genome is denoted c-Abl.

Philadelphia chromosome is a genetic abnormality in chromosome 22 formed by the t(9,22) reciprocal chromosome translocation, resulting in a fusion gene denoted BCR-ABL1. This fusion gene contains the ABL1 gene from chromosome 9 and part of the BCR gene. The tyrosine kinase activity of the ABL1 protein is normally tightly regulated, however, the BCR domains in the fusion gene result in constitutive activation of the ABL1 kinase. However, the binding domains of BCR-ABL and c-ABL are identical.

Activation of c-Abl has been implicated in various diseases, notably cancer. For instance, the presence of the BCR-ABL mutation is strongly linked to chronic myeloid leukaemia (CML). It is also found in some instances of acute lymphocytic leukaemia (ALL) and acute lymphoblastic leukaemia (ALL). Nilotinib and Ponatinib are both c-Abl inhibitors that have been used in the treatment of chronic myeloid leukaemia (CML) and acute lymphocytic leukaemia (ALL). The range of leukaemias that may be treated by c-ABL inhibition include chronic myeloid leukaemia (CML), acute lymphoblastic leukaemia (ALL), acute myelogenous leukaemia (AML), mixed-phenotype acute leukaemia (MPAL), and central nervous system (CNS) metastases thereof.

Activation of c-Abl has also been implicated in neurodegenerative diseases. Neurodegenerative diseases may be characterised by progressive degeneration and ultimate death of neurons. Particular neurodegenerative diseases include amyotrophic lateral sclerosis (ALS) and Parkinson's disease (PD).

ALS is a fatal neurodegenerative disease caused by the progressive degeneration of motor neurons. It has been reported that c-Abl signalling activation contributes to neuronal apoptosis and that c-Abl inhibitors can prevent motor neuron death [Rojas et al. Frontiers in Cellular Neuroscience, 2015, 9, 203; Imamura et al. Science Translational Medicine, 2017].

Parkinson's disease (PD) is a progressive neurodegenerative disorder caused by a selective loss of dopaminergic neurons in the substantia nigra pars compacta. It has been reported that c-Abl is activated in the brain of patients with PD and that c-Abl inhibition can protect against dopamine neuronal loss [Pagan et al. Pharmacology Research & Perspectives, 2019; Karuppagounder et al. Scientific Reports, 2014, 4, 4874].

Activation of c-Abl has also been implicated in a wide range of other diseases including, but not limited to, prion diseases, viral infections, diabetes, inflammatory diseases such as pulmonary fibrosis, and skeletal or muscular dystrophies.

Viral infections can be mediated by ABL1 kinase activity, as in the case of pox-viruses and the Ebola virus. Gleevec® and Tasigna® have been shown to stop the release of Ebola viral particles from infected cells, in vitro (see for instance WO 2007/002441; Mayra et al. Productive Replication of Ebola Virus Is Regulated by the ABL1 Tyrosine Kinase Science translational medicine 2012, 4, 123ra24). Inhibition of the ABL kinase can therefore be expected to reduce the pathogen's ability to replicate.

In prion disease models, Gleevec® showed beneficial effects. It delayed prion neuroinvasion by inhibiting prion propagation from the periphery to the CNS (Yun et al. The tyrosine kinase inhibitor imatinib mesylate delays prion neuroinvasion by inhibiting prion propagation in the periphery J Neurovirol. 2007, 13, 328-37). Gleevec® and ABL deficiency induced cellular clearance of PrPSc in prion-infected cells (Ertmer et al. The tyrosine kinase inhibitor STI571 induces cellular clearance of PrPSc in prion-infected cells J. Biol. Chem. 2004 279, 41918-27). Therefore, ABL1 inhibitors represent a valid therapeutic approach for the treatment of prion diseases, such as Creutzfeldt-Jacob disease (CJD).

X-linked recessive Emery-Dreifuss muscular dystrophy is caused by mutations of emerin, a nuclear-membrane protein with roles in nuclear architecture, gene regulation and signalling. A study has shown that emerin is tyrosine-phosphorylated directly by ABL1 in cell models, and that the phosphorylation status of emerin changes emerin binding to other proteins such as BAF. This, in turn, may explain the mislocalization of mutant emerin from nuclear to cytosolic compartments and consequently changes in downstream effector and signal integrator for signalling pathway(s) at the nuclear envelope (Tifft et al. Tyrosine phosphorylation of nuclear-membrane protein emerin by SRC, ABL1 and other kinases J. Cell Sci. 2009, 122, 3780-90). Changes in emerin-lamin interactions during both mitosis and interphase are of relevance for the pathology of muscular dystrophies. In addition, results from another study demonstrate that Gleevec® attenuates skeletal muscle dystrophy in mdx mice (Huang et al. Imatinib attenuates skeletal muscle dystrophy in mdx mice FASEB J. 2009, 23, 2539-48). Therefore, ABL1 inhibitors also represent therapeutic approaches for treatment of skeletal and muscular dystrophies.

Furthermore, ABL1 kinase plays a role in inflammation and oxidative stress, two mechanisms that are implicated in a variety of human diseases ranging from acute CNS diseases, such as stroke and traumatic brain or spinal cord injuries, chronic CNS diseases, such as Alzheimer's, Parkinson's, Huntington's and motoneuron diseases, to non-CNS inflammatory and autoimmune diseases, such as diabetes, pulmonary fibrosis.

For example, Gleevec® prevents fibrosis in different preclinical models of systemic sclerosis and induces regression of established fibrosis (Akhmetshina et al. Treatment with imatinib prevents fibrosis in different preclinical models of systemic sclerosis and induces regression of established fibrosis Arthritis Rheum. 2009, 60, 219-24) and it shows antifibrotic effects in bleomycin-induced pulmonary fibrosis in mice (Aono et al. Imatinib as a novel antifibrotic agent in bleomycin-induced pulmonary fibrosis in mice Am. J. Respir. Crit. Care Med. 2005, 171, 1279-85). Another study showed that both imatinib and nilotinib attenuated bleomycin-induced acute lung injury and pulmonary fibrosis in mice (Rhee et al. Effect of nilotinib on bleomycin-induced acute lung injury and pulmonary fibrosis in mice. Respiration 2011, 82, 273-87). Although in these studies the authors were focusing on the implication the mechanism related to PDGFRs, of interest, in the study by Rhee et al. (Respiration. 2011, 82, 273-87), nilotinib which is a more potent c-ABL inhibitor than imatinib showed superior therapeutic antifibrotic effects, thus supporting the therapeutic applicability of c-ABL inhibitors for treatment of human diseases with pulmonary inflammation. In another study, exposure of mice to hyperoxia increased ABL1 activation which is required for dynamin 2 phosphorylation and reactive oxygen species production and pulmonary leak (Singleton et al. Dynamin 2 and c-Abl are novel regulators of hyperoxia-mediated NADPH oxidase activation and reactive oxygen species production in caveolin-enriched microdomains of the endothelium J. Biol. Chem. 2009, 284, 34964-75).

In view of the above there is an unmet need for new compounds that may be used in the treatment and prevention of medical conditions in which inhibition of c-ABL is beneficial, such as neurodegenerative diseases (i.e. ALS and PD) and cancer (especially leukaemias).

### DISCLOSURE OF THE INVENTION

It has been found that compounds of Formula (I) may inhibit c-ABL and therefore treat or prevent the above medical conditions. Further, they have certain beneficial properties leading to increased potential for use as a drug compared to known compounds. This may be in terms of their efficacy, efflux profile (P-pg and/or BCRP), microsomal stability, free brain level at Cₘₐₓ, solubility, selectivity profiles, such as kinase selectivity, low hERG inhibitory activity, safety profile, and/or other notable pharmacokinetic properties.

Consequently, the first aspect of the invention relates to a compound of Formula (I),

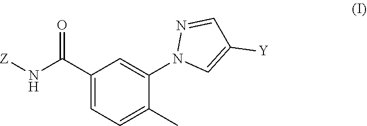

or a pharmaceutically acceptable salt, solvate, hydrate, tautomer, optical isomer, N-oxide, and/or prodrug thereof, wherein

Y is 5- or 6-membered heteroaryl optionally substituted with one or more substituents independently selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR¹R², —OR³, halo, and oxo;
(ii) halo, nitro, —CN, —C(O)NR⁴R⁵, —NR⁴R⁵, —C(O)OR⁶, —C(O)R⁶, and —OH; and
(iii) C₆-C₁₀ aryl, C₁-C₉ heteroaryl, and C₁-C₉ heterocycle, each of which is optionally substituted with one or more substituents independently selected from halo, C₁-C₆ alkyl, and C₁-C₆ haloalkyl;

Z is a 5- or 6-membered heteroaryl optionally substituted with one or more substituents selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR⁷R⁸, —OR⁹, halo, and oxo; and
(ii) halo, nitro, —CN, —C(O)NR¹⁰R¹¹, —NR¹⁰R¹¹, —C(O)OR¹², —C(O)R¹², and —OH; and

each of R¹ to R¹² is independently selected from the group consisting of H, C₁-C₆ alkyl, and C₁-C₆ haloalkyl; or

R¹ and R² and/or R⁴ and R⁵ may be taken together with the nitrogen atom to which they are respectively attached to form a 4- to 6-membered monocyclic heterocyclic ring that is optionally substituted with one or more substituents independently selected from halo, C₁-C₆ alkyl, and C₁-C₆ haloalkyl.

These compounds are compounds of the invention.

Without wishing to be bound by theory, the surprising beneficial properties of the compounds of the invention may be attributed, in part, to the pyrazole group connected to Group Y in the compound of Formula (I). It has been unexpectedly found that compounds that comprise the pyrazole group, and in particular regioisomeric form in the compounds of Formula (I), have high BAF3/ABL inhibitory activity (indicated by low IC₅₀ values) and low P-gp efflux and BCRP efflux, making them particularly useful in the treatment of certain diseases and conditions. This is in comparison with a range of similar compounds, including compounds that contain a triazole in place of the pyrazole attached to Group Y in Formula (I), and different regioisomers of said pyrazole. It is noted that P-gp/BCRP efflux is unpredictable and must be established empirically. Overcoming the challenges associated with delivering therapeutic agents with low susceptibility to efflux presents a major challenge to treatment of many disorders, particularly disorders affecting the brain.

P-glycoprotein 1 (P-gp) also known as multidrug resistance protein 1 (MDR1) or ATP-binding cassette sub-family B member 1 (ABCB1) or cluster of differentiation 243 (CD243) is an important protein of the cell membrane that pumps many foreign substances out of cells. It is an ATP-dependent efflux pump with broad substrate specificity, and is likely evolved as a defence mechanism against harmful substances. P-gp is extensively distributed and expressed in the capillary endothelial cells composing the blood-brain barrier (as well as the blood-testis barrier) where it pumps xenobiotics (such as toxins or drugs) back into the capillaries. It is also present in the intestinal epithelium where it pumps xenobiotics back into the intestinal lumen, in liver cells where it pumps them into bile ducts, and in the cells of the proximal tubule of the kidney where it pumps them into urinary filtrate (in the proximal tubule).

Breast cancer resistance protein (BCRP), also known as ATP-binding cassette super-family G member 2 (ABCG2). It is a membrane-associated protein that transports various molecules across extra- and intra-cellular membranes, and has been shown to play protective roles in blocking absorption at the blood-brain barrier as well as the apical membrane of the intestine, the blood-testis barrier, and the membranes of hematopoietic progenitor and other stem cells.

In cases where the drug target is located within the central nervous system, it is usually preferable that compounds are not substrates for these efflux transporters in order to prevent restriction to the periphery with limited CNS exposure.

As used herein, “5- or 6-membered heteroaryl” is an aromatic monocyclic hydrocarbon ring in which at least one, such as 1, 2, 3 or 4, ring atom is a heteroatom. Each 5- or 6-membered heteroaryl, as it appears in the definition of group Y and group Z, is independently selected, i.e. they may be the same or different. Examples of groups that form the basis of 5- or 6-membered heteroaryls useful as in the compounds of the invention include, but are not limited to, optionally substituted pyrrolyl, pyrazolyl, imidazolyl, triazolyl, tetrazolyl, oxazolyl, isoxazolyl, thiazolyl, isothiazolyl, oxadiazolyl, thiadiazolyl, pyridyl, pyridazinyl, pyrimidinyl, pyrazinyl, and triazinyl.

In a preferred feature of the invention, the 5- or 6-membered heteroaryl of group Y is selected from the group consisting of optionally substituted pyrrolyl, pyrazolyl, imidazolyl, triazolyl, tetrazolyl, oxazolyl, isoxazolyl, thiazolyl, isothiazolyl, oxadiazolyl, thiadiazolyl, pyridyl, pyridazinyl, pyrimidinyl, pyrazinyl, and triazinyl. More preferably it is selected from optionally substituted pyrrolyl, pyrazolyl, imidazolyl, pyridyl, pyridyl, pyridazinyl, pyrimidinyl, and pyrazinyl, and most preferably optionally substituted pyrimidinyl, pyrazolyl and pyridyl. In these cases, the optionally substitution is with one or more substituents independently selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR¹R², —OR³, halo, and oxo;
(ii) halo, nitro, —CN, —C(O)NR⁴R⁵, —NR⁴R⁵, —C(O)OR⁶, —C(O)R⁶, and —OH; and
(iii) C₆-C₁₀ aryl, C₁-C₉ heteroaryl, and C₁-C₉ heterocycle, each of which is optionally substituted with one or more substituents independently selected from halo, C₁-C₆ alkyl, and C₁-C₆ haloalkyl.

Preferable examples of 5- and 6-membered heteroaryls for group Het may be in the following regioisomeric forms

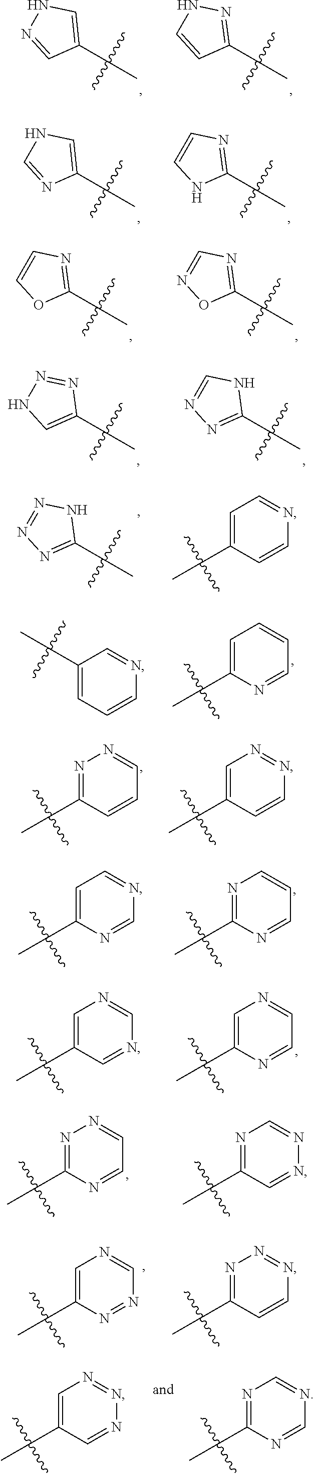

or a tautomer thereof, with each group being optionally substituted, including substitution of the H attached to a N atom. More preferably they may be the following regioisomeric forms

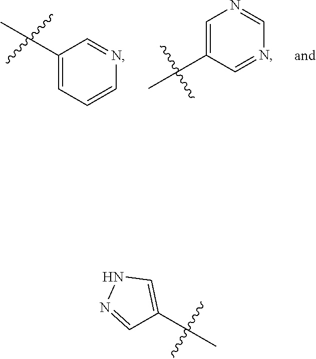 [SMILES: *c1cccnc1 ; *c1cncnc1 ; *c1cn[nH]c1]

or a tautomer thereof, with each group being optionally substituted, including substitution of the H attached to a N atom.

In particular feature of the first aspect of the invention, Y is selected from one of the following groups

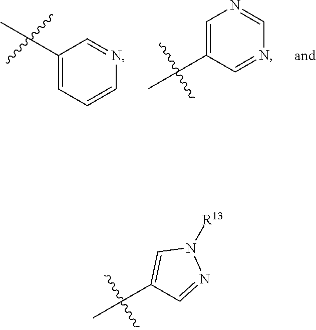

each of which being optionally substituted with one or more substituents independently selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR¹R², —OR³, halo, and oxo;
(ii) halo, nitro, —CN, —C(O)NR⁴R⁵, —NR⁴R⁵, —C(O)OR⁶, —C(O)R⁶, and —OH; and
(iii) C₆-C₁₀ aryl, C₁-C₉ heteroaryl, and C₁-C₉ heterocycle, each of which is optionally substituted with one or more substituents independently selected from halo, C₁-C₆ alkyl, and C₁-C₆ haloalkyl; and
   wherein R¹³ is selected from the group selected from the group consisting of H, C₁-C₆ alkyl and C₁-C₆ haloalkyl.

Particularly preferable compounds of the invention are those in which group Y is optionally substituted with one or more substituents independently selected from the group consisting of
(i) C₁-C₆ alkyl, and C₁-C₆ alkoxy, each of which is optionally substituted with one or more substituents independently selected from —NR¹R², —OR³, halo, and oxo; and
(ii) halo, —C(O)NR⁴R⁵, —NR⁴R⁵, —C(O)OR⁶, —C(O)R⁶, and —OH.

Even more preferable, group Y is optionally substituted with one or more substituents independently selected from the group consisting of —OH, halo, C₁-C₆ alkyl, C₁-C₆ alkoxy, C₁-C₆ haloalkyl, and —NR⁴R⁵, such as —OH, C₁-C₆ alkyl, C₁-C₆ alkoxy, C₁-C₆ haloalkyl, and —NR⁴R⁵. Most preferably, R⁴ and R⁵ are independently selected from the group consisting of H, C₁-C₃ alkyl, and C₁-C₃ haloalkyl.

As mentioned, the 5- or 6-membered heteroaryl of group Z may be different from that of group Y. It is preferable that group Z is selected from the group consisting of optionally substituted pyrrolyl, pyrazolyl, imidazolyl, triazolyl, tetrazolyl, oxazolyl, isoxazolyl, thiazolyl, isothiazolyl, oxadiazolyl, thiadiazolyl, pyridyl, pyridazinyl, pyrimidinyl, pyrazinyl, and triazinyl, preferably optionally substituted pyrrolyl, pyrazolyl, imidazolyl, triazolyl, thiazolyl, oxazolyl, pyridyl, pyridazinyl, pyrimidinyl, pyrazinyl, and triazinyl, most preferably pyridazinyl, and pyridyl, each of which is optionally substituted with one or more substituents selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR⁷R⁸, —OR⁹, halo, and oxo; and
(ii) halo, nitro, —CN, —C(O)NR¹⁰R¹¹, —NR¹⁰R¹¹, —C(O)OR¹², —C(O)R¹², and —OH.

In this regard, it is even more preferable that group Z is a 6-membered heteroaryl, and in particular it is selected from one of the following groups

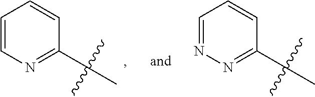 [SMILES: *c1ccccn1 ; *c1cccnn1]

each of which being optionally substituted with one or more substituents selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR⁷R⁸, —OR⁹, halo, and oxo; and
(ii) halo, nitro, —CN, —C(O)NR¹⁰R¹¹, —NR¹⁰R¹¹, —C(O)OR¹², —C(O)R¹², and —OH.

A particularly useful set of substituents for group Z is one or more substituents independently selected from the group consisting of halo, —CN, and C₁-C₆ haloalkyl, wherein halo is preferably fluoro, such as halo and C₁-C₆ haloalkyl, wherein halo is preferably fluoro.

Without wishing to be bound by theory, it may be particularly advantageous to include a hydrophobic group as a substituent on the 5- or 6-membered heteroaryl of group Z, as this may increase interaction with a hydrophobic pocket of c-Abl, thereby increasing binding affinity. It is most preferable that the 5- or 6-membered heteroaryl of group Z is substituted with one or more groups selected from halo and C₁-C₆ haloalkyl, wherein halo is preferably fluoro. Most preferably group Z is 6-membered heteroaryl, such as one selected from

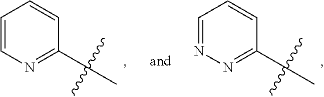 [SMILES: *c1ccccn1 ; *c1cccnn1]

each of which is substituted with one or more groups selected from halo and C₁-C₆ haloalkyl, wherein halo is preferably fluoro.

The heteroaryl of group Z may have particular advantages over corresponding aryl analogues, such as higher BAF3/ABL inhibitory activity (indicated by lower IC₅₀ values) and lower P-gp efflux and/or BCRP efflux, making them particularly useful in the treatment of certain diseases and conditions.

Particularly useful compounds of Formula (I) are those in which Y is optionally substituted with one or more substituents independently selected from the group consisting of —OH, halo, C₁-C₃ alkyl, C₁-C₃ alkoxy, C₁-C₃ haloalkyl, and —NR⁴R⁵;

R⁴ and R⁵ are independently selected from the group consisting of H, C₁-C₃ alkyl, and C₁-C₃ haloalkyl; or

R⁴ and R⁵ may be taken together with the nitrogen atom to which they are respectively attached to form a 5- to 6-membered monocyclic heterocyclic ring that is optionally substituted with one or more substituents independently selected from halo, C₁-C₃ alkyl, and C₁-C₃ haloalkyl; Preferably, R⁴ and R⁵ are independently selected from the group consisting of H, C₁-C₃ alkyl, and C₁-C₃ haloalkyl;

Z is selected from one of the following groups

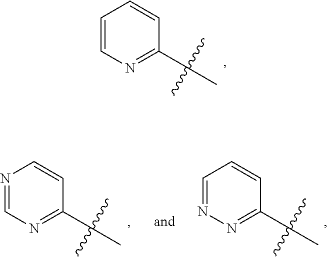 [SMILES: *c1ccccn1 ; *c1ccncn1 ; *c1cccnn1]

preferably

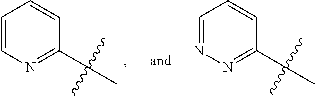 [SMILES: *c1ccccn1 ; *c1cccnn1]

each of which is optionally substituted with one or more substituents independently selected from the group consisting of halo, —CN and C₁-C₃ haloalkyl, preferably —CF₃. In this case, Y may be substituted with a substituent selected from the group consisting of —NH(C₁-C₃)alkyl and

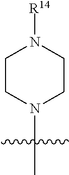

In view of the above, particularly useful compounds of Formula (I) are those in which Y is optionally substituted with one or more substituents independently selected from the group consisting of —OH, C₁-C₃ alkyl, C₁-C₃ alkoxy, C₁-C₃ haloalkyl, and —NR⁴R⁵;

R⁴ and R⁵ are independently selected from the group consisting of H, C₁-C₃ alkyl, and C₁-C₃ haloalkyl; or

R⁴ and R⁵ may be taken together with the nitrogen atom to which they are respectively attached to form a 5- to 6-membered monocyclic heterocyclic ring that is optionally substituted with one or more substituents independently selected from halo, C₁-C₃ alkyl, and C₁-C₃ haloalkyl; Preferably, R⁴ and R⁵ are independently selected from the group consisting of H, C₁-C₃ alkyl, and C₁-C₃ haloalkyl;

Z is selected from one of the following groups

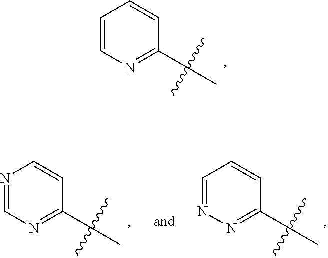 [SMILES: *c1ccccn1 ; *c1ccncn1 ; *c1cccnn1]

preferably

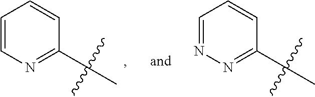 [SMILES: *c1ccccn1 ; *c1cccnn1]

each of which is optionally substituted with one or more substituents independently selected from the group consisting of halo and C₁-C₃ haloalkyl, preferably —CF₃. In this case, Y may be substituted with a substituent selected from the group consisting of —NH(C₁-C₃)alkyl and

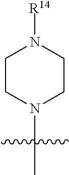

wherein R¹⁴ is selected from the group consisting of H, C₁-C₃ alkyl and C₁-C₃ haloalkyl.

Specific compounds of Formula (I) include
4-Methyl-3-[4-(3-pyridyl)pyrazol-1-yl]-N-[4-(trifluoromethyl)-2-pyridyl]benzamide;
4-Methyl-3-(4-pyrimidin-5-ylpyrazol-1-yl)-N-[4-(trifluoromethyl)-2-pyridyl]benzamide;
4-Methyl-3-[4-(3-pyridyl)pyrazol-1-yl]-N-[5-(trifluoromethyl)pyridazin-3-yl]benzamide;
3-[4-(1,5-Dimethylpyrazol-4-yl)pyrazol-1-yl]-4-methyl-N-[4-(trifluoromethyl)-2-pyridyl]benzamide;
4-Methyl-3-[4-[2-(methylamino)pyrimidin-5-yl]pyrazol-1-yl]-N-[4-(trifluoromethyl)-2-pyridyl]benzamide;
4-Methyl-3-[4-[5-(4-methylpiperazin-1-yl)-3-pyridyl]pyrazol-1-yl]-N-[4-(trifluoromethyl)-2-pyridyl]benzamide;
4-Methyl-3-[4-(5-methylpyridin-3-yl)-1H-pyrazol-1-yl]-N-[4-(trifluoromethyl)pyridin-2-yl]benzamide;
3-[4-(5-Fluoropyridin-3-yl)-1H-pyrazol-1-yl]-4-methyl-N-[4-(trifluoromethyl)pyridin-2-yl]benzamide;
3-[4-(5-methoxypyridin-3-yl)-1H-pyrazol-1-yl]-4-methyl-N-[4-(trifluoromethyl)pyridin-2-yl]benzamide; and
N-[6-cyano-4-(trifluoromethyl)pyridin-2-yl]-4-methyl-3-[4-(pyridin-3-yl)-1H-pyrazol-1-yl]benzamide,
   or a pharmaceutically acceptable salt, solvate, hydrate, tautomer, optical isomer, N-oxide, and/or prodrug thereof.

The compounds of the invention may include isotopically-labelled and/or isotopically-enriched forms of the compounds. The compounds of the invention herein may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl.

The compounds of the invention may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Throughout the present disclosure, a given chemical formula or name shall also encompass all pharmaceutically acceptable salts, solvates, hydrates, N-oxides, and/or prodrug forms thereof. It is to be understood that the compounds of the invention include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g. having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis- and trans-geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The term “prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

Another object of the present invention relates to the compounds of the invention for use in therapy.

The compounds of the invention are useful as inhibitors of c-ABL. As such, they are useful in the treatment or prevention of medical conditions (conditions or diseases) in which inhibition of c-ABL is beneficial. There is therefore provided a method of for the treatment or prevention of a disease or condition responsive to c-ABL inhibition comprising administering a therapeutically effective amount of a compound of the invention to a subject. Whilst the compounds of the invention may be suitable to prevent a range of diseases and conditions, it is preferable that they are used to treat said diseases and conditions. Therefore, it is preferred that the method is for the treatment of a disease or condition, and therefore the method comprises administering a therapeutically effective amount of a compound of the invention to a subject in need thereof.

The term “treatment” as used herein may include prophylaxis of the named disorder or condition, or amelioration or elimination of the disorder once it has been established. The term “prevention” refers to prophylaxis of the named disorder or condition.

The range of diseases and conditions treatable or preventable by c-ABL inhibition is well known. The compounds of the invention therefore may be used to treat or prevent this range of diseases or conditions. This includes neurodegenerative disorders, cancers, prion diseases, viral infections, diabetes, inflammatory diseases such as pulmonary fibrosis, or a skeletal or muscular dystrophy. Preferably, the disease is a neurodegenerative disorder or a cancer.

Treatable or preventable neurodegenerative disorders include, but are not limited to, Alzheimer disease, Down's syndrome, frontotemporal dementia, progressive supranuclear palsy, Pick's disease, Niemann-Pick disease, Parkinson's disease, Huntington's disease (HD), dentatorubropallidoluysian atrophy, Kennedy's disease, and spinocerebellar ataxia, fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis (ALS), ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

Of the treatable or preventable neurodegenerative disorders, most notable are amyotrophic lateral sclerosis (ALS) and Parkinson's disease. Most preferably the neurodegenerative disorder is ALS.

Treatable or preventable cancers include, but are not limited to, leukaemia.

Of the treatable or preventable cancers, most notable are chronic myeloid leukaemia (CML), acute lymphoblastic leukaemia (ALL), acute myelogenous leukaemia (AML), and mixed-phenotype acute leukaemia (MPAL), or any central nervous system (CNS) metastases thereof. Most preferably the cancer is CML or ALL.

The invention thus includes the use of the compounds of the invention in the manufacture of a medicament for the treatment or prevention of a disease or condition, such as the above-mentioned neurodegenerative disorders and cancers. The invention also relates to the compounds of the invention for use in the treatment of a disease or condition, such as the above-mentioned neurodegenerative disorders and cancers.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

The invention provides a method of monitoring treatment progress. The method includes the step of determining a level of diagnostic marker (Marker) (e.g. any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

A level of Marker or Marker activity in a subject may be determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, is ELISA, radiolabeling/assay techniques, blotting/chemiluminescence methods, real-time PCR, and the like.

For clinical use, the compounds disclosed herein are formulated into pharmaceutical compositions (or formulations) for various modes of administration. It will be appreciated that compounds of the invention may be administered together with a physiologically acceptable carrier, excipient, and/or diluent (i.e. one, two, or all three of these). The pharmaceutical compositions disclosed herein may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration. Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### DEFINITIONS

The term “substituted” means that the group to which it refers has one or more hydrogen atoms substituted for a different group. For instance, “substituted pyrazolyl” refers to a monovalent radical of pyrazole with one or more hydrogens attached to the ring being replaced with another group.

The term “heteroatom” means O, N, or S. Typically, it is preferred that the heteroatom or heteroatoms in the 5- or 6-membered heteroaryl is nitrogen.

“Optional” or “optionally” means that the subsequently described event or circumstance may, but need not, occur, and that the description includes instances where the event or circumstance occurs and instances in which it does not.

The term “C₁-C₆ alkyl” denotes a straight, branched or cyclic or partially cyclic alkyl group having from 1 to 6 carbon atoms, i.e. 1, 2, 3, 4, 5 or 6 carbon atoms. For the “C₁-C₆ alkyl” group to comprise a cyclic portion it should be formed of 3 to 6 carbon atoms. For parts of the range “C₁-C₆ alkyl” all subgroups thereof are contemplated, such as C₁-C₅ alkyl, C₁-C₄ alkyl, C₁-C₃ alkyl, C₁-C₂ alkyl, C₁ alkyl, C₂-C₆ alkyl, C₂-C₅ alkyl, C₂-C₄ alkyl, C₂-C₃ alkyl, C₂ alkyl, C₃-C₆ alkyl, C₃-C₅ alkyl, C₃-C₄ alkyl, C₃ alkyl, C₄-C₆ alkyl, C₄-C₅ alkyl, C₄ alkyl, C₅-C₆ alkyl, C₅ alkyl, and C₆ alkyl. Examples of “C₁-C₆ alkyl” include methyl, ethyl, n-propyl, isopropyl, cyclopropyl, n-butyl, isobutyl, sec-butyl, t-butyl, cyclobutyl, cyclopropylmethyl, and straight, branched or cyclic or partially cyclic pentyl and hexyl etc.

When a term denotes a range, for instance “1 to 6 carbon atoms” in the definition of C₁-C₆ alkyl, each integer is considered to be disclosed, i.e. 1, 2, 3, 4, 5 and 6.

The term “C₂-C₆ alkenyl” denotes a straight, branched or cyclic or partially cyclic alkyl group having at least one carbon-carbon double bond, and having from 2 to 6 carbon atoms. The alkenyl group may comprise a ring formed of 3 to 6 carbon atoms. For parts of the range “C₂-C₆ alkenyl” all subgroups thereof are contemplated, such as C₂-C₅ alkenyl, C₂-C₄ alkenyl, C₂-C₃ alkenyl, C₂ alkenyl, C₃-C₆ alkenyl, C₃-C₅ alkenyl, C₃-C₄ alkenyl, C₃ alkenyl, C₄-C₆ alkenyl, C₄-C₅ alkenyl, C₄ alkenyl, C₅-C₆ alkenyl, C₅ alkenyl, and C₆ alkenyl. Examples of “C₂-C₆ alkenyl” include 2-propenyl, 2-butenyl, 3-butenyl, 2-methyl-2-propenyl, 2-hexenyl, 5-hexenyl, 2,3-dimethyl-2-butenyl.

The term “C₂-C₆ alkynyl” denotes a straight, branched or cyclic or partially cyclic alkyl group having at least one carbon-carbon triple bond, and having from 2 to 6 carbon atoms. The alkynyl group may comprise a ring formed of 3 to 6 carbon atoms. For parts of the range “C₂-C₆ alkynyl” all subgroups thereof are contemplated, such as C₂-C₅ alkynyl, C₂-C₄ alkynyl, C₂-C₃ alkynyl, 0 2 alkynyl, C₃-C₆ alkynyl, C₃-C₅ alkynyl, C₃-C₄ alkynyl, C₃ alkynyl, C₄-C₆ alkynyl, C₄-C₅ alkynyl, C₄ alkynyl, C₅-C₆ alkynyl, C₅ alkynyl, and C₆ alkynyl. Examples of “C₂-C₆ alkynyl” include 2-propynyl, 2-butynyl, 3-butynyl, 2-pentynyl, 3-methyl-4-pentynyl, 2-hexynyl, 5-hexynyl etc.

The term “C₁-C₆ alkoxy” denotes —O-(C₁-C₆alkyl) in which a C₁-C₆ alkyl group is as defined above and is attached to the remainder of the compound through an oxygen atom. Examples of “C₁-C₆ alkoxy” include methoxy, ethoxy, n-propoxy, isopropoxy, n-butoxy, isobutoxy, sec-butoxy, t-butoxy and straight- and branched-chain pentoxy and hexoxy.

The term “halo” means a halogen atom, and is preferably, F, Cl, Br and I, more preferably F and Cl, and most preferably F.

The term “C₁-C₆ haloalkyl” means a C₁-C₆ alkyl group in which one or more hydrogen atoms are replaced with a halo atoms, preferably F.

The term “oxo” denotes a double bond to an oxygen atom (═O). This typically forms a ketone or aldehyde group.

The term “C₆-C₁₀ aryl” denotes an aromatic monocyclic or fused bicyclic hydrocarbon ring comprising 6 to 10 ring atoms. Examples of “C₆-C₁₀ aryl” groups include phenyl, indenyl, naphthyl, and naphthalene.

The term “C₁-C₉ heteroaryl” denotes an aromatic monocyclic or fused bicyclic heteroaromatic ring system having 5 to 10 ring atoms in which 1 to 9 of the ring atoms are carbon and one or more of the ring atoms are selected from nitrogen, sulphur, and oxygen. Examples of “C₁-C₉ heteroaryl” include furyl, pyrrolyl, thienyl, oxazolyl, isoxazolyl, imidazolyl, thiazolyl, isothiazolyl, pyridinyl, pyrimidinyl, tetrazolyl, quinazolinyl, indolyl, indolinyl, isoindolyl, isoindolinyl, pyrazolyl, pyridazinyl, pyrazinyl, quinolinyl, quinoxalinyl, thiadiazolyl, benzofuranyl, 2,3-dihydrobenzofuranyl, 1,3-benzodioxolyl, 1,4-benzodioxinyl, 2,3-dihydro-1,4-benzodioxinyl, benzothiazolyl, benzimidazolyl, benzothiadiazolyl, benzotriazolyl and chromanyl.

The term “C₁-C₉ heterocycle” denotes a non-aromatic monocyclic or fused bicyclic ring system having 5 to 10 ring atoms containing 1 to 9 carbon atoms and one or more of the ring atoms are selected from nitrogen, sulfur, and oxygen. When present, the sulfur atom may be in an oxidized form (i.e. the diradicle of S═O or the diradical of O═S═O). The ring system may be fully saturated or partially unsaturated. Examples of “C₁-C₉ heterocycle” include piperidinyl, tetrahydropyranyl, tetrahydrofuranyl, oxetanyl, azepinyl, azetidinyl, pyrrolidinyl, morpholinyl, imidazolinyl, imidazolidinyl, thiomorpholinyl, pyranyl, dioxanyl, piperazinyl, homopiperazinyl, and 5,6-dihydro-4H-1,3-oxazin-2-yl. Similarly, a “4- to 6-membered monocyclic heterocyclic ring” denotes a non-aromatic monocyclic ring having 4, 5 or 6 ring atoms and one or more of the ring atoms are selected from nitrogen, sulfur and oxygen. Examples of this include piperidinyl, tetrahydropyranyl, tetrahydrofuranyl, pyrrolidinyl amongst others listed above.

“An effective amount” refers to an amount of a compound of the invention that confers a therapeutic effect on the treated subject. The therapeutic effect may be objective (i.e. measurable by some test or marker) or subjective (i.e. subject gives an indication of or feels an effect).

As used herein, the terms “administration” or “administering” mean a route of administration for a compound disclosed herein. Exemplary routes of administration include, but are not limited to, oral, intravenous, intraperitoneal, intraarterial, and intramuscular. The preferred route of administration can vary depending on various factors, e.g. the components of the pharmaceutical composition comprising a compound disclosed herein, site of the potential or actual disease and severity of disease.

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. It is preferred that the subject is human.

Compounds of the invention may be disclosed by the name or chemical structure. If a discrepancy exists between the name of a compound and its associated chemical structure, then the chemical structure prevails.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilise the present invention to its fullest extent. All references and publications cited herein are hereby incorporated by reference in their entirety.

### PREPARATION OF COMPOUNDS OF THE INVENTION

The compounds of Formula (I) disclosed herein may be prepared by, or in analogy with, conventional methods. Appropriate reaction conditions for the individual reaction steps are known to a person skilled in the art. The necessary starting materials for preparing the compounds of Formula (I) are either commercially available, or may be prepared by methods known in the art.

The compounds of Formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

Particular experimental procedures for examples of the invention are described below. The processes may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl(triphenylmethyl). The methods described below may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

### EXAMPLES AND INTERMEDIATE COMPOUNDS

### Experimental Methods

All reagents were commercial grade and were used as received without further purification, unless otherwise specified. Reagent grade solvents were used in all cases.

LC-MS and UPLC data was recorded under the following conditions:

### Method A

Waters Aquity system BEH-C18, 1.7 μm, 2.1′ 50 mm, 40° C., 0.5 μL injection, 0.4 mL/min. 0% MeCN (+0.1% aq. NH₃+5% H₂O) in H₂O (+0.1% NH₃+5% MeCN) for 0.2 min, 0-100% over 3.3 min, hold for 1 min, re-equilibrate 1.0 min, 200-400 nm.

### Method B

Agilent 1100 (quaternary pump) XBridge-C18, 5 μm, 4.6×50 mm, 25° C., 2 mL/min, 5 μL injection, 5% MeCN in H₂O (+10 mM ammonium formate), gradient 5-95% over 3.5 min, hold for 1 min, 200-400 nm.

### Method C

Waters Aquity system CSH-C18, 1.7 μm, 2.1×50 mm, 40° C., 0.5 μL injection, 0.4 mL/min. 0% MeCN (+0.1% formic acid+5% H₂O) in H₂O (+0.1% formic acid+5% MeCN) for 0.2 min, 0-100% over 3.3 min, hold for 1 min, 200-400 nm.

### Method D

Shimadzu LCMS-2020 system with PDA: SPD-M20A and MS: LCMS-2020 detectors. Column: Halo, 2.0*30 mm, mobile phase A: H₂O (0.1% formic acid), mobile phase B: MeCN; Flow rate: 1.5 mL/min; Gradient as described per compound, in 1.5 min

### Method E

Shimadzu LCMS-2020 system with PDA: SPD-M20A and MS: LCMS-2020 detectors. Column: Halo, 3.0*30 mm, mobile phase A: H₂O (0.1% formic acid), mobile phase B: MeCN (0.1% formic acid); Flow rate: 1.5 mL/min; Gradient as described per compound, in 1.5 min.

### Method F

Shimadzu LCMS-2020 system with PDA: SPD-M20A and MS: LCMS-2020 detectors. Column: L-column 3 C18, 3.0*30 mm, mobile phase A: H₂O (5 mM NH₄HCO₃), mobile phase B: MeCN; Flow rate: 1.5 mL/min; Gradient: 30-70% B in 1.5 min.

### Intermediate 1

### Methyl 4-methyl-3-pyrazol-1-yl-benzoate

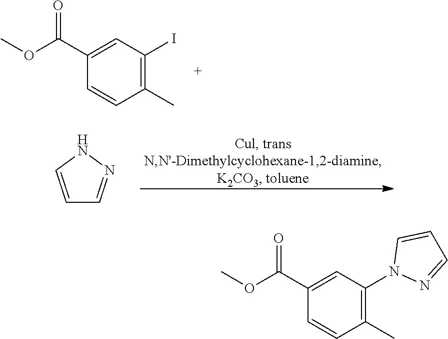

A round bottom flask (RBF) was charged with pyrazole (2.50 g, 36.7 mmol), CuI (699 mg, 3.7 mmol), K₂CO₃ (10.66 g, 77.1 mmol) and PhMe (100 mL) and the mixture degassed. Methyl 3-iodo-4-methylbenzoate (12.17 g, 44.1 mmol) and trans N,N′-dimethylcyclohexane-1,2-diamine (1.16 mL, 7.3 mmol) were added and the reaction mixture was heated overnight at 110° C. Two additional portions of CuI (699 mg, 3.7 mmol) and trans N,N′-dimethylcyclohexane-1,2-diamine (1.16 mL, 7.3 mmol) were added and reaction mixture heated overnight at 120° C. after each addition. The reaction was allowed to cool to rt, filtered and concentrated in vacuo. The crude residue was purified on silica (100% heptane to EtOAc:heptane, 3:17) to give the title compound (4.60 g, 55%) as an off white solid. UPLC (Method A) 2.61 min, 95.6%, [M+H]⁺=217.3.

### Intermediate 2

### Methyl 3-(4-bromopyrazol-1-yl)-4-methyl-benzoate

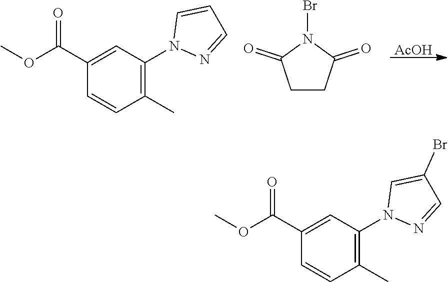

In four separate microwave (MW) vials, a solution of Intermediate 1 (425 mg, 1.97 mmol), N-bromosuccinimide (385 mg, 2.16 mmol) in AcOH (14 mL) was heated under MW irradiation for 15 min at 150° C. The MW vials were combined and the solvent was removed under reduced pressure. The resulting residue was purified on silica (EtOAc:heptane, 1:9 to 2:3) to afford the title compound (2.10 g, 89%) as an off-white solid. UPLC (Method A) 3.50 min, 98.5%, [M+H]⁺=295.0/297.0 (Br79/81).

### Intermediate 3

### Methyl 4-methyl-3-[4-(3-pyridyl)pyrazol-1-yl]benzoate

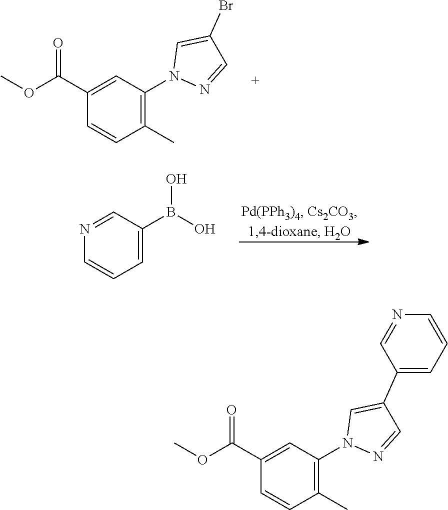

To a degassed solution of Intermediate 2 (1.30 g, 4.40 mmol), pyridin-3-ylboronic acid (812 mg, 6.61 mmol) and Cs₂CO₃ (4.31 g, 13.23 mmol) in 1,4-dioxane (55 mL) and H₂O (9.0 mL) was added Pd(PPh₃)₄ (255 mg, 0.22 mmol). The reaction mixture was heated to 100° C. overnight. The resulting mixture was allowed to cool to rt, extracted with EtOAc (30 mL), washed with aq. NaHCO₃ (10 mL), dried over Na₂SO₄, and concentrated in vacuo to give a crude oil. The oil was purified on silica (100% heptane to EtOAc:heptane, 1:1) to give the title compound (1.10 g, 82%). LCMS (Method B) 2.76 min, 100%, [M+H]⁺=294.5.

### Intermediate 4

### [4-Methyl-3-[4-(3-pyridyl)pyrazol-1-yl]benzoyl]oxypotassium

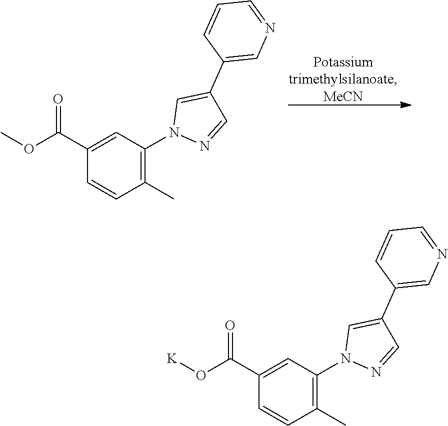

To a solution of Intermediate 3 (330 mg, 1.13 mmol) in MeCN (20 mL) was added potassium trimethylsilanoate (289 mg, 2.25 mmol) and the mixture stirred at rt for 16 h. The reaction mixture was filtered and triturated with further MeCN (˜5.0 mL) and TBME (˜5.0 mL) to give the title compound (380 mg, quant.) as a colourless solid after drying. UPLC (Method A) 1.80 min, 100%, [M+H]⁺=280.1.

### Intermediate 5

### 3-(4-Bromopyrazol-1-yl)-4-methyl-N-[4-(trifluoromethyl)-2-pyridyl]benzamide

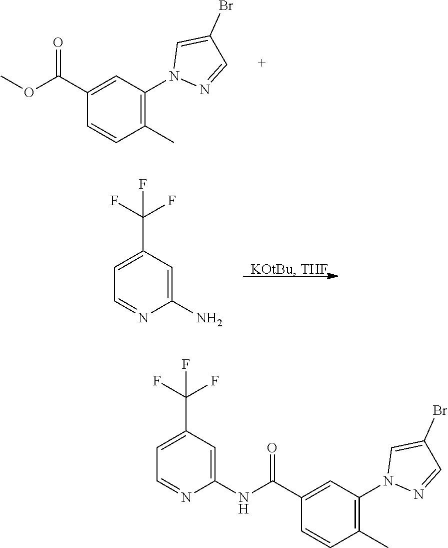 [SMILES: COC(=O)c1ccc(C)c(-n2cc(Br)cn2)c1 ; Nc1cc(C(F)(F)F)ccn1 ; Cc1ccc(C(=O)Nc2cc(C(F)(F)F)ccn2)cc1-n1cc(Br)cn1]

To a solution of (140 mg, 0.47 mmol) and 2-amino-4-(trifluoromethyl)pyridine (76.9 mg, 0.47 mmol) in THF (4.7 mL) was added ^{t}BuOK (20% sol THF, 1.39 mL, 2.37 mmol) and the reaction mixture was stirred at room temperature overnight. The reaction was quenched with brine (15 mL) and extracted with EtOAc (3×15 mL). The combined organics were dried over MgSO₄ and concentrated to give the crude compound. The oil was purified on silica (100% heptane to EtOAc:heptane, 1:4) to give the title compound (40 mg, 19%) as an off white solid. UPLC (Method A) 3.39 min, 94.8%, [M+H]⁺=425.2/427.1 (Br79/81).

### Intermediate 6

### N-methyl-5-(4,4,5,5-tetramethyl-1,3,2-dioxaborolan-2-yl)pyrimidin-2-amine

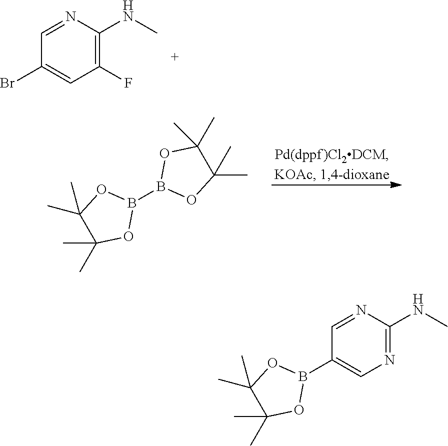

To a degassed solution of 5-bromo-N-methylpyrimidin-2-amine (500 mg, 2.66 mmol), bis(pinacolato)diboron (810 mg, 3.19 mmol) and KOAc (783 mg, 7.98 mmol) in 1,4-dioxane (5.0 mL) was added Pd(dppf)Cl₂·DCM (109 mg, 0.13 mmol) and the mixture stirred at 80° C. overnight. Brine (15 mL) was added to the reaction mixture and it was extracted with EtOAc (3×20 mL). Combined organics were dried over Na₂SO₄ and concentrated in vacuo to provide crude product. The crude was further purified on silica (EtOAc:heptane, 1:4 to 4:1) to provide the title compound (460 mg, 72%) as a yellow oily solid. UPLC (Method A) 0.68 min, 97.7%, [M+H]⁺=236.2.

### Intermediate 7

### 1-(5-Bromo-3-pyridyl)-4-methyl-piperazine

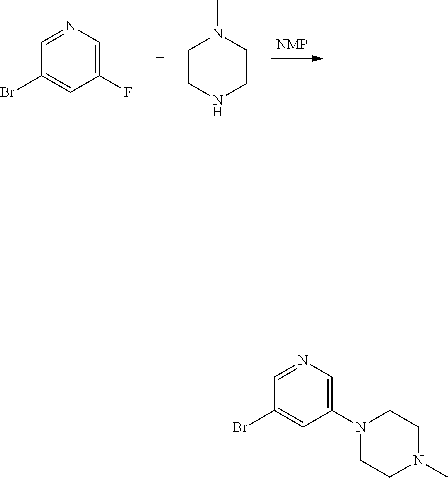

To a solution of 3-bromo-5-fluoropyridine (1.00 g, 5.7 mmol) in NMP (15 mL) was added 1-methylpiperazine (3.36 mL, 28.4 mmol) and the reaction heated to 150° C. in the microwave for 2 h. The reaction mixture was partitioned between EtOAc (150 mL) and sat. aq. NaHCO₃ solution (150 mL). The layers were separated and the aqueous layer further extracted with EtOAc (150 mL). The combined organics were washed with H₂O (150 mL) and brine (150 mL) before drying over MgSO₄. The organics were concentrated to give the crude product. The resulting material was purified by cationic exchange resin SCX-2 to afford the title compound (1.18 g, 80%) as a colourless oil. UPLC (Method A) 2.54 min, 98.2%, [M+H]⁺=256.1/258.1 (Br79/81).

### Intermediate 8

### 1-Methyl-4-[5-(4,4,5,5-tetramethyl-1,3,2-dioxaborolan-2-yl)-3-pyridyl]piperazine

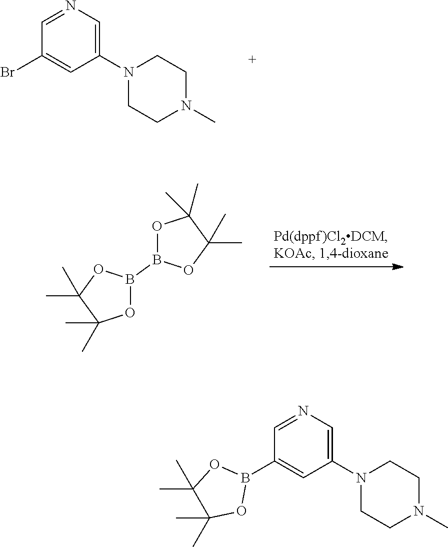

To a degassed solution of Intermediate 7 (500 mg, 1.95 mmol), bis(pinacolato)diboron (595 mg, 2.34 mmol) and KOAc (575 mg, 5.86 mmol) in 1,4-dioxane (5.0 mL) was added Pd(dppf)Cl₂·DCM (80 mg, 0.10 mmol) and the mixture stirred at 80° C. overnight. The reaction was filtered through a syringe filter and used in the next step without further purification.

### Intermediate 9

### 3-Nitro-5-(trifluoromethyl)benzoyl chloride

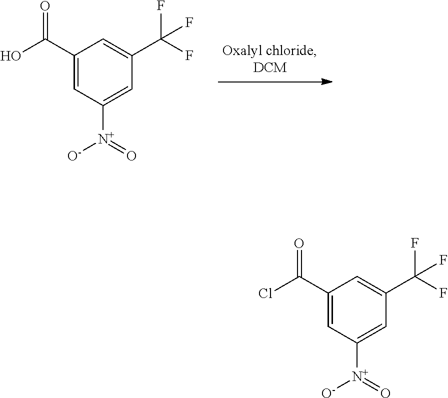 [SMILES: O=C(O)c1cc([N+](=O)[O-])cc(C(F)(F)F)c1 ; O=C(Cl)c1cc([N+](=O)[O-])cc(C(F)(F)F)c1]

To a suspension of 3-nitro-5-(trifluoromethyl)benzoic acid (233 mg, 0.99 mmol) in DCM (2.5 mL) was added oxalyl chloride (0.43 mL, 4.96 mmol). The reaction was stirred for 2 h at rt under N₂. The volatiles were evaporated in vacuo to give provide the title compound (300 mg, 98%) as an orange oil. The compound was used crude in the next step. UPLC (Method A) 3.05 min, 82.4% (derivatised by addition of MeOH. Methyl ester does not ionise).

### Intermediate 10

### [3-Nitro-5-(trifluoromethyl)phenyl]-pyrrolidin-1-yl-methanone

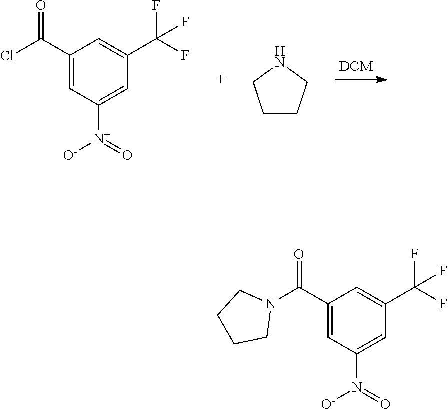 [SMILES: O=C(Cl)c1cc([N+](=O)[O-])cc(C(F)(F)F)c1 ; C1CCNC1 ; O=C(c1cc([N+](=O)[O-])cc(C(F)(F)F)c1)N1CCCC1]

To a suspension of Intermediate 9 (252 mg, 0.99 mmol) in DCM (2.5 mL) was added pyrrolidine (0.25 mL, 3.00 mmol) at 0° C. The reaction was stirred overnight at rt under N₂. The reaction mixture was diluted with EtOAc (10 mL), the organic layer was washed with H₂O (10 mL), sat. brine (10 mL), dried over MgSO₄ and filtered. The filtrate was concentrated under reduced pressure to give the title compound (300 mg, 90%) as an orange oil. The compound was used crude in the next step. UPLC (Method A) 2.72 min, 85.7%, [M+H]⁺=289.2.

### Intermediate 12

### [3-Amino-5-(trifluoromethyl)phenyl]-pyrrolidin-1-yl-methanone

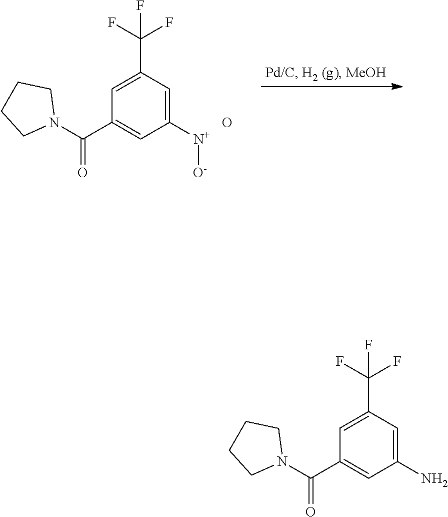

To a solution of Intermediate 10 (286 mg, 0.99 mmol) in MeOH (2.0 mL) was added 5% Pd/C (31 mg, 0.29 mmol). The vessel was sealed and stirred under an atmosphere of H₂ (at 1 bar) at rt overnight. The mixture was filtered through a pad of Dicalite eluting with MeOH and the solvent removed under reduced pressure to afford the title compound (210 mg, 72%) as an off-white white solid. UPLC (Method A) 2.37 min, 87.9%, [M+H]⁺=259.3.

### Intermediate 12

### 3-(Pyrrolidin-1-ylmethyl)-5-(trifluoromethyl)aniline

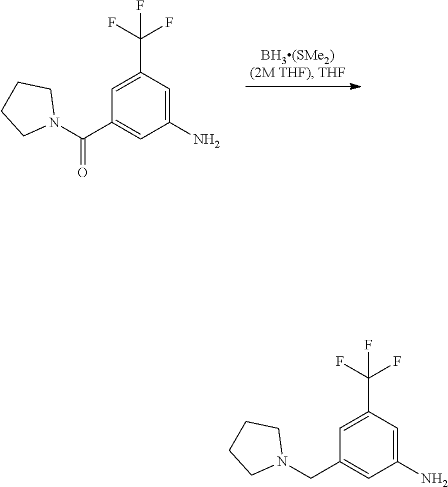 [SMILES: Nc1cc(C(=O)N2CCCC2)cc(C(F)(F)F)c1 ; Nc1cc(CN2CCCC2)cc(C(F)(F)F)c1]

To a solution of Intermediate 11 (210 mg, 0.80 mmol) in THF (5.0 mL) was added dropwise BH₃·(SMe₂) 2M THF (1.22 mL, 2.41 mmol) at 0° C., and the mixture was stirred at rt for 1 h and then under reflux for 3 h. After cooling the reaction solution to rt, 6N HCl (2.5 mL) was added. After stirring for 30 min, the mixture was stirred at reflux for 2 h. After cooling the reaction solution to 0° C., 8N aq. NaOH (2.5 mL) was added. The mixture was diluted with H₂O (15 mL) and extracted with EtOAc (3×10 mL). The organic layer was washed with sat. brine (15 mL), dried over MgSO₄ and filtered. The filtrate was concentrated under reduced pressure. The crude residue was purified on silica (DCM:heptane, 4:1) to give the title compound (140 mg, 54%) as an off white solid. UPLC (Method A) 3.03 min, 76.7%, [M+H]⁻=243.3.

### Intermediate 13

### Morpholino-[3-nitro-5-(trifluoromethyl)phenyl]methanone

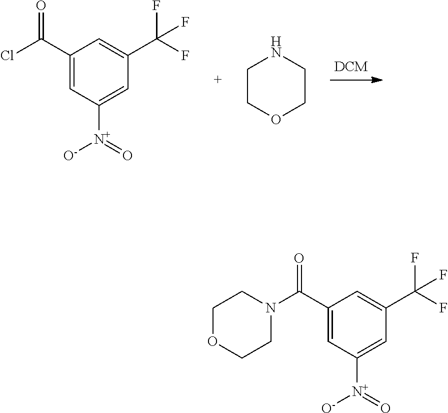 [SMILES: O=C(Cl)c1cc([N+](=O)[O-])cc(C(F)(F)F)c1 ; C1COCCN1 ; O=C(c1cc([N+](=O)[O-])cc(C(F)(F)F)c1)N1CCOCC1]

To a suspension of Intermediate 9 (252 mg, 0.99 mmol) in DCM (2.5 mL) was added morpholine (261 mg, 3.00 mmol) at 0° C. The reaction was stirred overnight at rt under N₂. The reaction mixture was diluted with EtOAc (10 mL), and the organic layer was washed with H₂O (10 mL) and sat. brine (10 mL), dried over MgSO₄ and filtered. The filtrate was concentrated under reduced pressure to give the title compound (300 mg, 98%) as an orange oil. The compound was used crude, without further purification. UPLC (Method A) 2.48 min, 97.1%, [M+H]⁺=305.3.

### Intermediate 14

### [3-Amino-5-(trifluoromethyl)phenyl]-morpholino-methanone

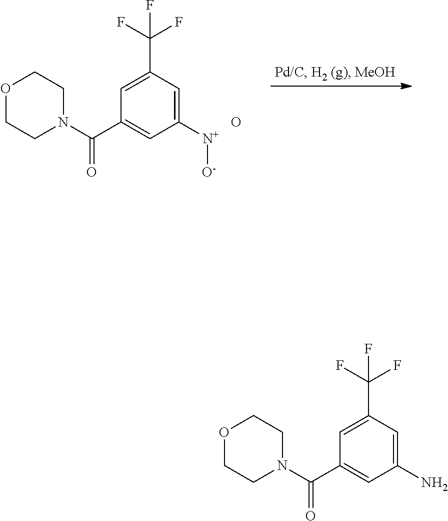

To a solution of Intermediate 13 (302 mg, 0.99 mmol) in MeOH (2.0 mL) was added 5% Pd/C (31 mg, 0.29 mmol). The vessel was sealed and stirred under an atmosphere of H₂ (at 1 bar) at rt overnight. The mixture was filtered through a pad of Dicalite eluting with MeOH and the solvent removed under reduced pressure to afford the title compound (220 mg, 69%) as an off-white solid. UPLC (Method A) 2.13 min, 85.6%, [M+H]⁺=275.3.

### Intermediate 15

### 3-(Morpholinomethyl)-5-(trifluoromethyl)aniline

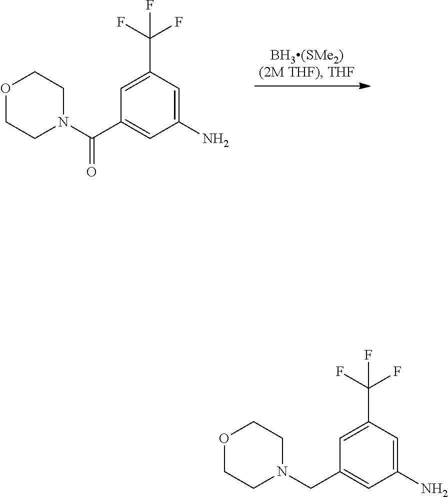 [SMILES: Nc1cc(C(=O)N2CCOCC2)cc(C(F)(F)F)c1 ; Nc1cc(CN2CCOCC2)cc(C(F)(F)F)c1]

To a solution of Intermediate 14 (220 mg, 0.80 mmol) in THF (5.0 mL) was added dropwise BH₃·(SMe₂) 2M THF (1.20 mL, 2.41 mmol) at 0° C., and the mixture was stirred at rt for 1 h and then under reflux for 3 h. After cooling the reaction solution to rt, 6N HCl (2.5 mL) was added. After stirring for 30 min, the mixture was stirred under reflux for 2 h. After cooling the reaction solution to 0° C., 8N aq. NaOH (2.5 mL) was added. The mixture was diluted with H₂O (15 mL) and extracted with EtOAc (3×10 mL). The organic layer was washed with sat. brine (15 mL), dried over MgSO₄ and filtered. The filtrate was concentrated under reduced pressure. The crude residue was purified on silica (DCM:heptane, 4:1) to give the title compound (120 mg, 53%) as an off white solid. UPLC (Method A) 2.41 min, 91.4%, [M+H]⁻=259.3.

### Intermediate 16

### Methyl 3-azido-4-methyl-benzoate

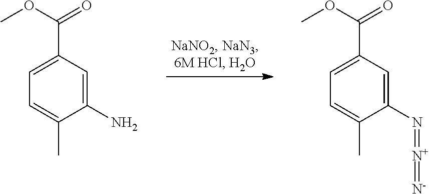

Methyl 3-amino-4-methylbenzoate (4.96 g, 30.0 mmol) was dissolved in HCl (6.0M in H₂O, 24.0 mL, 144.0 mmol) at rt, cooled to 0° C., treated with a solution of NaNO₂ (2.10 g in 30 mL of H₂O, 30.0 mmol) and stirred for 10 min at −5° C. Sodium azide (2.05 g in 30 mL of H₂O, 31.5 mmol) was added slowly, and the mixture was stirred at rt for 2 h under N₂. The mixture was diluted with EtOAc (70 mL), and the organic layer was washed with brine (30 mL), dried over Na₂SO₄, and concentrated to give the title compound (5.30 g, 92%) as a yellow oil. UPLC (Method C) 2.41 min, 99.4%, no ionisation.

### Intermediate 17

### Methyl 4-methyl-3-[4-(3-pyridyl)triazol-1-yl]benzoate

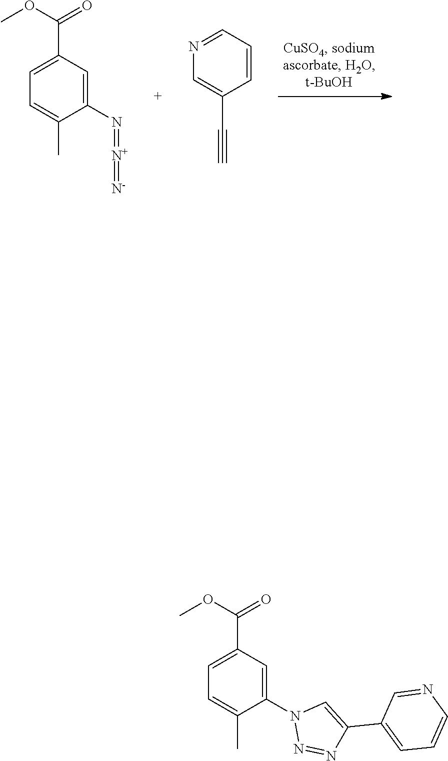

Intermediate 16 (700 mg, 3.66 mmol) and 3-ethynylpyridine (378 mg, 3.66 mmol) were dissolved in a mixture of tBuOH (7.0 mL) and H₂O (7.0 mL) at rt under N₂. CuSO₄ (584 mg, 3.66 mmol) and sodium ascorbate (1.45 g, 7.32 mmol) were added and the mixture was stirred at rt overnight. The reaction mixture was poured into a sat. solution of EDTA disodium salt in H₂O (150 mL) and EtOAc (30 mL) and the resulting mixture was stirred for 6 h at rt. The fractions were separated and the aqueous phase was extracted with EtOAc (2×30 mL). The combined organics were washed with sat. EDTA disodium salt solution (30 mL), brine (30 mL), dried over Na₂SO₄ and concentrated to give the crude product. This was further purified on silica (100% DCM to 100% EtOAc) to provide the title compound (335 mg, 31%) as an off-white solid. UPLC (Method C) 2.42 min, 99.5%, [M+H]⁺=295.2.

### Intermediate 10

### 4-Methyl-3-[4-(3-pyridyl)triazol-1-yl]benzoic acid

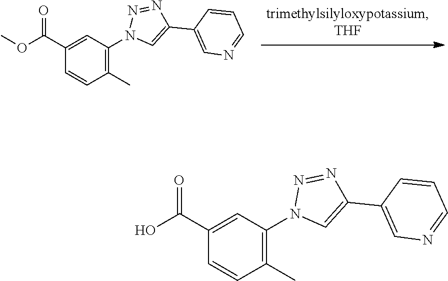 [SMILES: COC(=O)c1ccc(C)c(-n2cc(-c3cccnc3)nn2)c1 ; Cc1ccc(C(=O)O)cc1-n1cc(-c2cccnc2)nn1]

To a solution of Intermediate 17 (830 mg, 2.82 mmol) in THF (50 mL) was added KOTMS (650 mg, 5.07 mmol). The reaction was stirred at rt for 24 h. The resultant residue was suspended in MTBE (20 mL) and filtered. The cake was dissolved in H₂O (5.0 mL) and acidified to pH2 using HCl (2.0M, aq.). The precipitate was collected by filtration, dried, azeotroped with MeCN (2×10 mL) and dried to give the title compound (850 mg, 108%) with some H₂O still present. UPLC (Method A) 1.89 min, 100%, [M+H]⁺=281.1.

### Intermediate 19

### Methyl 4-methyl-3-[3-(3-pyridyl)pyrazol-1-yl]benzoate

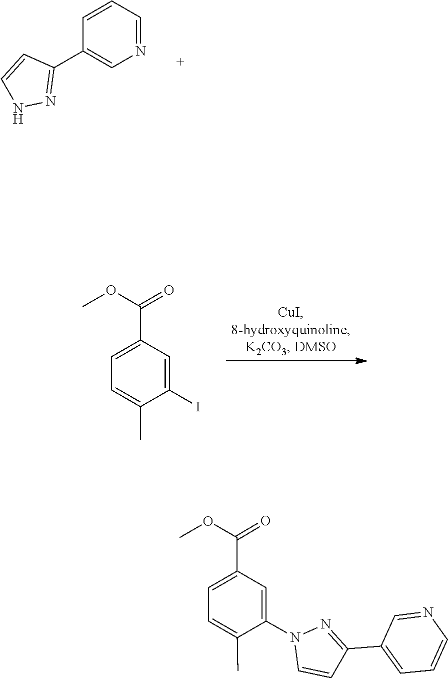 [SMILES: c1cncc(-c2cc[nH]n2)c1 ; COC(=O)c1ccc(C)c(I)c1 ; COC(=O)c1ccc(C)c(-n2ccc(-c3cccnc3)n2)c1]

To a MW vial was suspended 3-(1H-pyrazol-3-yl)pyridine (50.0 mg, 0.34 mmol), methyl 3-iodo-4-methylbenzoate (95.1 mg, 0.34 mmol), 8-hydroxyquinoline (50.0 mg, 0.34 mmol), K₂CO₃ (71.4 mg, 0.52 mmol) and CuI (65.6 mg, 0.34 mmol) in DMSO (3.0 mL). The reaction mixture was heated under MW irradiation for 2 h at 150° C. The reaction was diluted with H₂O (5.0 mL) and extracted with TBME (3×10 mL). Combined organics were washed with brine (10 mL), dried over Na₂SO₄ and concentrated in vacuo to give the title compound (60.0 mg, 45%) as a yellow oil. UPLC (Method A) 3.0 min, 76.4%, [M+H]⁺=294.2.

### Intermediate 20

### 4-Methyl-3-[3-(3-pyridyl)pyrazol-1-yl]benzoic acid

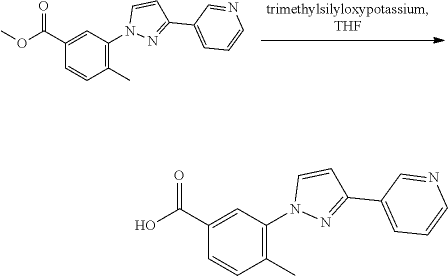 [SMILES: COC(=O)c1ccc(C)c(-n2ccc(-c3cccnc3)n2)c1 ; Cc1ccc(C(=O)O)cc1-n1ccc(-c2cccnc2)n1]

To a solution of Intermediate 19 (270 mg, 0.60 mmol) in THF (10 mL) was added KOTMS (231 mg, 1.80 mmol). The reaction was stirred at rt overnight. To a separate solution of Intermediate 19 (60 mg, 0.16 mmol) in THF (2.0 mL) was added KOTMS (36 mg, 0.28 mmol). The reaction was stirred at rt overnight. Both reactions were combined and the volatiles were evaporated in vacuo. The resulting solid was suspended in TBME (20 mL) and put in an ultrasound bath for 5 min. The solvent was filtered off and the solid was dried. The solid was dissolved in a H₂O/DMSO mixture (1:1, 1 mL) and the residue was further purified on silica (Biotage Isolera, reverse phase, MeCN:H₂O, 1:9 to 4:1, both eluents containing 0.1 Vol % NH₃) to provide the title compound (140 mg, 65%) as a white solid. UPLC (Method A) 2.08 min, 100%, [M+H]⁺=280.1. [The correct regioisomer was confirmed by NOESY].

### Intermediate 21

### 4-Methyl-3-[3-(3-pyridyl)pyrazol-1-yl]benzamide

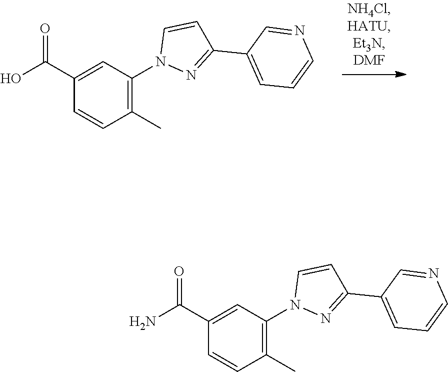 [SMILES: Cc1ccc(C(=O)O)cc1-n1ccc(-c2cccnc2)n1 ; Cc1ccc(C(N)=O)cc1-n1ccc(-c2cccnc2)n1]

To a suspension of Intermediate 20 (140 mg, 0.50 mmol), NH₄Cl (80 mg, 1.50 mmol) and Et₃N (0.22 mL, 1.55 mmol) in DMF (10 mL) was added HATU (286 mg, 0.75 mmol). The reaction was stirred at rt overnight under N₂. The reaction was quenched with H₂O (10 mL) and TBME was added (10 mL). The mixture was put in an ultrasound bath for 5 min and the solvents were filtered off to give crude. The residue was further purified on silica (MeOH:DCM, 1:49 to 1:19) to provide the title compound (90.0 mg, 65%). UPLC (Method A) 2.40 min, 100%, [M+H]⁺=279.1.

### Example 1

### 4-Methyl-3-[4-(3-pyridyl)pyrazol-1-yl]-N-[4-(trifluoromethyl)-2-pyridyl]benzamide

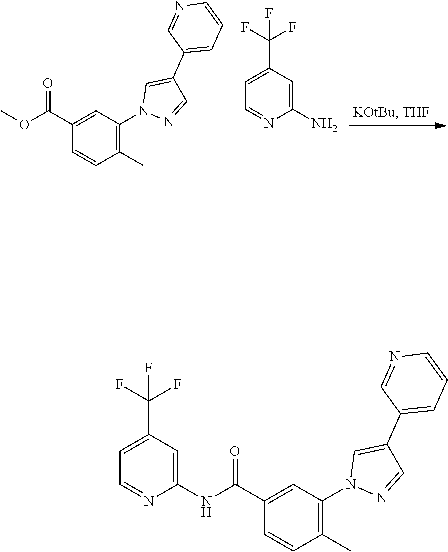

To a solution of Intermediate 3 (1.09 g, 3.70 mmol) and 2-amino-4-(trifluoromethyl)pyridine (500 mg, 3.08 mmol) in THF (35 mL) was added KOtBu (1.7M in THF, 9.07 mL, 15.4 mmol) and the reaction mixture was stirred at rt overnight under N₂. The reaction mixture was concentrated, diluted with H₂O and the resulting precipitate collected on a frit and triturated with TBME. The precipitate was re-dissolved in EtOAc (50 mL), washed with K₂CO₃ (2×30 mL), the organics dried over Na₂SO₄ and concentrated to give a beige powder. The powder was triturated with MeCN:TBME and collected on a frit to give the title compound (292 mg, 22%) as an off-white solid. UPLC (Method A) 3.32 min, 100%, [M+H]⁺=424.2.

### Example 2

### 4-Methyl-3-[4-(3-pyridyl)pyrazol-1-yl]-N-[5-(trifluoromethyl)pyridazin-3-yl]benzamide

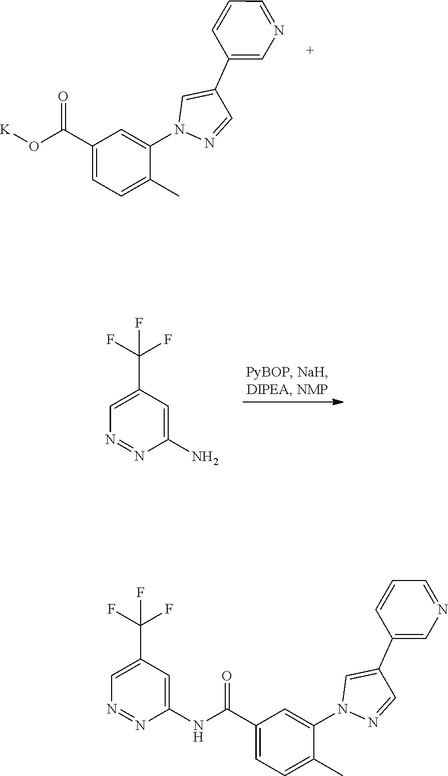

To a solution of 5-(trifluoromethyl)pyridazin-3-amine (51.4 mg, 0.32 mmol) in NMP (2.0 mL) was added NaH (60% w/w in mineral oils, 18.9 mg, 0.47 mmol) and the mixture stirred at rt for 20 min. This was then added to a stirred solution of Intermediate 4 (100 mg, 0.32 mmol), DIPEA (0.08 mL, 0.47 mmol) and PyBOP (197 mg, 0.38 mmol) in NMP (1.0 mL) and the resulting dark brown reaction mixture stirred at rt for 2 h. This was partitioned between H₂O (40 mL), sat. aq. NH₄Cl (40 mL) and TBME (50 mL). The aqueous was extracted with further TBME (1×50 mL) and the combined TBME layers washed with 10% K₂CO₃ solution (2×50 mL), dried over MgSO₄ and concentrated to give a pale brown oily residue that was purified on silica (Biotage Isolera, reverse phase, MeCN:H₂O, 2:3 to 3:2, both eluents containing 0.1 Vol % NH₃) to give the title compound (9.8 mg, 7%) as an off-white solid. UPLC (Method A) 3.00 min, 97.0%, [M+H]⁺=425.2.

### Example 3

### 4-Methyl-3-(4-pyrimidin-5-ylpyrazol-1-yl)-N-[4-(trifluoromethyl)-2-pyridyl]benzamide

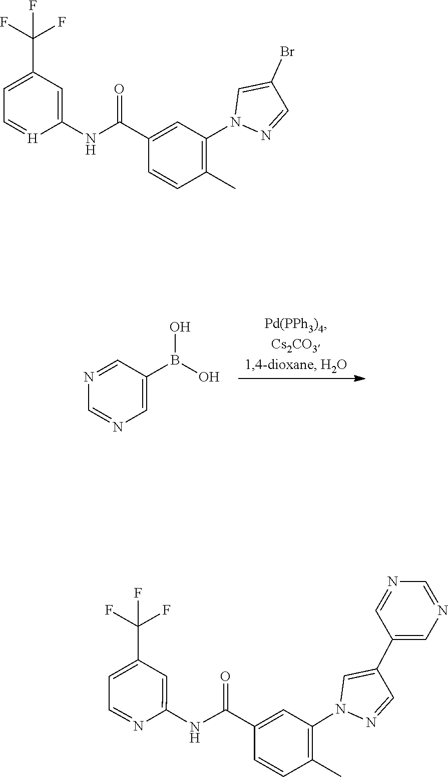

To a degassed solution of Intermediate 5 (30.0 mg, 0.07 mmol), pyrimidine-5-boronic acid (13.1 mg, 0.11 mmol) and Cs₂CO₃ (69.0 mg, 0.21 mmol) in dioxane (0.9 mL) and H₂O (0.2 mL) was added Pd(PPh₃)₄ (4.1 mg, 3.50 μmol). The reaction mixture was then heated to 95° C. overnight. The product was extracted with EtOAc (10 mL) and washed with aq. NH₄Cl (10 mL) and aq. NaHCO₃ (10 mL) then brine (10 mL), dried over MgSO₄, and concentrated in vacuo to give the crude oil. The oil was purified on silica (Biotage Isolera, reverse phase, MeCN:H₂O, 1:9 to 4:1, both eluents containing 0.1 Vol % NH₃) to give the title compound (15.0 mg, 50%) as an off-white solid. UPLC (Method A) 3.15 min, 99.4%, [M+H]⁺=425.2.

### Example 4

### 3-[4-(1,5-Dimethylpyrazol-4-yl)pyrazol-1-yl]-4-methyl-N-[4-(trifluoromethyl)-2-pyridyl]benzamide

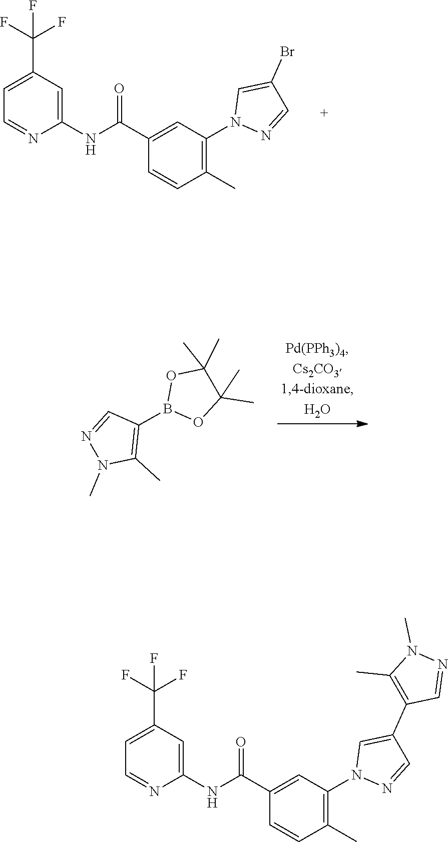 [SMILES: Cc1ccc(C(=O)Nc2cc(C(F)(F)F)ccn2)cc1-n1cc(Br)cn1 ; Cc1c(B2OC(C)(C)C(C)(C)O2)cnn1C ; Cc1ccc(C(=O)Nc2cc(C(F)(F)F)ccn2)cc1-n1cc(-c2cnn(C)c2C)cn1]

To a degassed solution of Intermediate 5 (127 mg, 0.30 mmol), 1,5-dimethyl-4-(4,4,5,5-tetramethyl-1,3,2-dioxaborolan-2-yl)pyrazole (100 mg, 0.45 mmol) and Cs₂CO₃ (292 mg, 0.90 mmol) in 1,4-dioxane:H₂O (4.0 mL, 3:1) was added Pd(PPh₃)₄ (17 mg, 0.01 mmol) and the reaction stirred at 100° C. overnight. H₂O (10 mL) was added to the reaction mixture and the mixture was extracted with EtOAc (3×10 mL). The combined organics were washed with brine (10 mL), dried over Na₂SO₄ and concentrated. The crude material was purified on silica (Biotage Isolera, reverse phase, MeCN:H₂O, 1:4 to 4:1, both eluents containing 0.1 Vol % NH₃). The residue was further purified via trituration (×3) with TBME (2.0 mL) and the residue dissolved in MeCN (5.0 mL) and freeze-dried to yield the title compound (11.2 mg, 8%) as off-white solid. UPLC (Method A) 3.30 min, 92.5%, [M+H]⁺=441.2

### Example 5

### 4-Methyl-3-[4-[2-(methylamino)pyrimidin-5-yl]pyrazol-1-yl]-N-[4-(trifluoromethyl)-2-pyridyl]benzamide

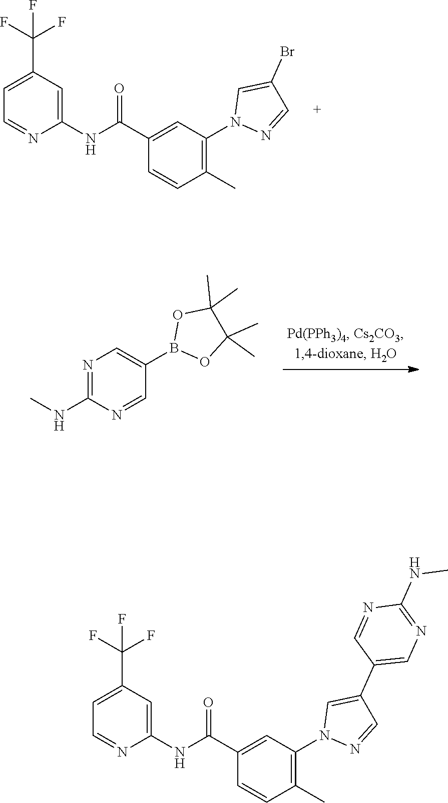

To a degassed solution of Intermediate 5 (100 mg, 0.24 mmol), N-methyl-5-(4,4,5,5-tetramethyl-1,3,2-dioxaborolan-2-yl)pyrimidin-2-amine (85 mg, 0.35 mmol) and Cs₂CO₃ (230 mg, 0.71 mmol) in 1,4-dioxane:H₂O (4.0 mL, 3:1) was added Pd(PPh₃)₄ (14 mg, 0.01 mmol) and the reaction stirred at 100° C. overnight. H₂O (10 mL) was added and the mixture extracted with EtOAc (3×10 mL). Combined organics were washed with brine (10 mL), dried over Na₂SO₄ and concentrated. The crude material was further purified on silica (Biotage Isolera, reverse phase, MeCN:H₂O, 1:4 to 4:1, both eluents containing 0.1 Vol % NH₃). The resulting solid was dissolved in MeCN (5.0 mL) and freeze-dried overnight to yield the title compound (16.4 mg, 15%) as colourless solid. UPLC (Method A) 3.23 min, 100%, [M+H]⁺=454.2

### Example 6

### 4-Methyl-3-[4-[5-(4-methylpiperazin-1-yl)-3-pyridyl]pyrazol-1-yl]-N-[4-(trifluoromethyl)-2-pyridyl]benzamide

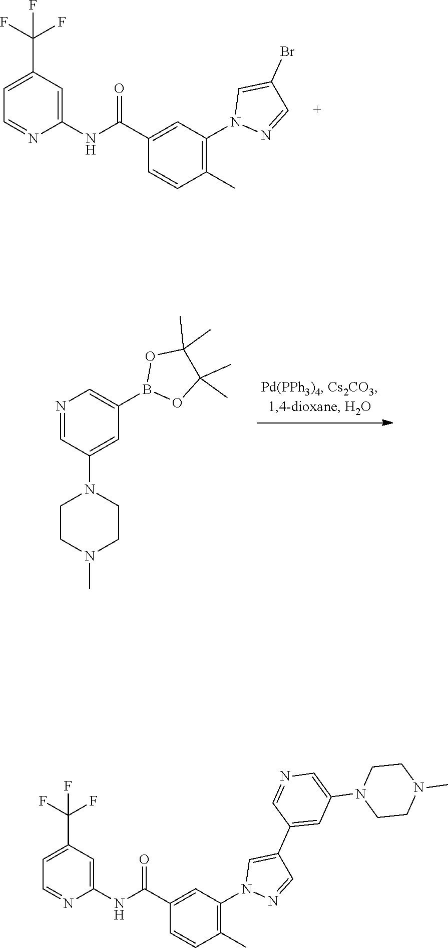 [SMILES: Cc1ccc(C(=O)Nc2cc(C(F)(F)F)ccn2)cc1-n1cc(Br)cn1 ; CN1CCN(c2cncc(B3OC(C)(C)C(C)(C)O3)c2)CC1 ; Cc1ccc(C(=O)Nc2cc(C(F)(F)F)ccn2)cc1-n1cc(-c2cncc(N3CCN(C)CC3)c2)cn1]

To a degassed crude solution of 1-methyl-4-[5-(4,4,5,5-tetramethyl-1,3,2-dioxaborolan-2-yl)-3-pyridyl]piperazine (107 mg, 0.35 mmol) in 1,4-dioxane (3.0 mL) was added Intermediate 5 (100 mg, 0.24 mmol), Cs₂CO₃ (230 mg, 0.71 mmol) and H₂O (1.0 mL). Pd(PPh₃)₄ (14 mg, 0.01 mmol) was added to the reaction mixture and the reaction stirred at 100° C. overnight and at rt for 3 days. H₂O (10 mL) was added and the mixture was extracted with EtOAc (3×10 mL). Combined organics were washed with brine (10 mL), dried over Na₂SO₄ and concentrated. The crude was further purified on silica (Biotage Isolera, reverse phase, MeCN:H₂O, 1:4 to 4:1, both eluents containing 0.1 Vol % NH₃). The resulting solid was further purified via SCX column and the resulting solid dissolved in MeCN (5.0 mL) and freeze-dried to yield the title compound (22.5 mg, 18%) as an off-white solid. UPLC (Method A) 3.26 min, 100%, [M+H]⁺=522.3.

### Example 7

### 4-Methyl-3-[4-(5-methylpyridin-3-yl)-1H-pyrazol-1-yl]-N-[4-trifluoromethyl)pyridin-2-yl]benzamide

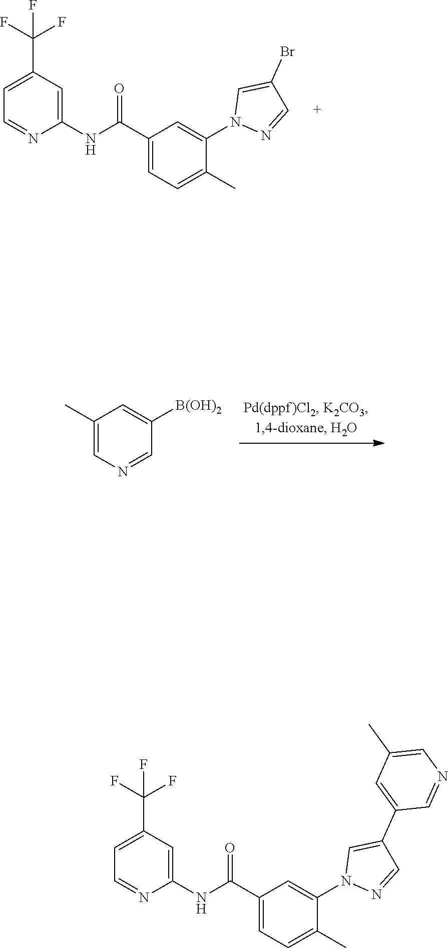

A mixture of Intermediate 5 (100 mg, 0.24 mmol), 5-methylpyridin-3-ylboronic acid (32 mg, 0.24 mmol), K₂CO₃ (65 mg, 0.47 mmol) and Pd(dppf)Cl₂ (38 mg, 0.05 mmol) in 1,4-dioxane:H₂O (4.0 mL, 3:1) was stirred at 100° C. for 2 h. The reaction was quenched with H₂O at rt and the organics were removed in vacuo. The resultant solution was extracted with EtOAc (2×40 mL). Combined organics were washed with brine (20 mL), dried over anhydrous Na₂SO₄, filtered and concentrated. The crude material was further purified by prep-HPLC (C18, H₂O (0.1% formic acid)/MeCN, 7:3 to 13:12) to yield the title compound (24 mg, 23%) as a white solid. LCMS (Method D) 1.40 min, [M+H]+=438.2, gradient 5-50% B. HRMS m/z: [M+H]⁺ calculated for C₂₃H₁₉F₃N₅O 438.1542; found 438.1544.

### Example 8

### 3-[4-(5-Fluoropyridin-3-yl)-1H-pyrazol-1-yl]-4-methyl-N-[4-(trifluoromethyl)pyridin-2-yl]benzamide

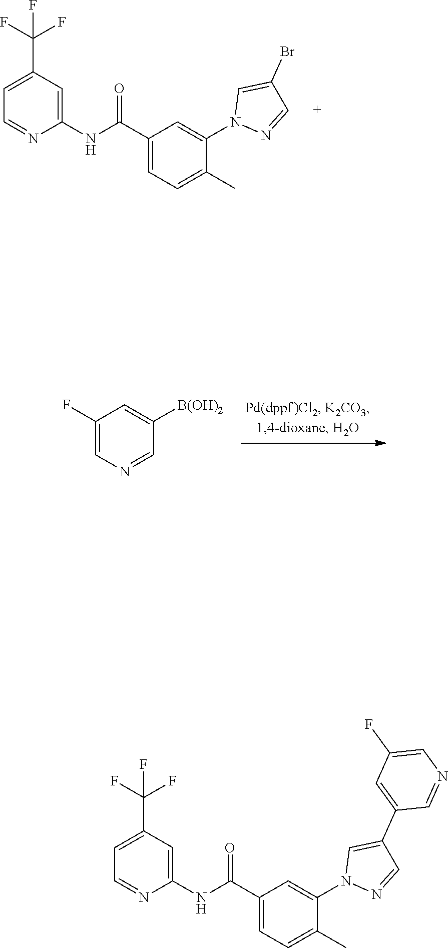 [SMILES: Cc1ccc(C(=O)Nc2cc(C(F)(F)F)ccn2)cc1-n1cc(Br)cn1 ; OB(O)c1cncc(F)c1 ; Cc1ccc(C(=O)Nc2cc(C(F)(F)F)ccn2)cc1-n1cc(-c2cncc(F)c2)cn1]

A mixture of Intermediate 5 (100 mg, 0.24 mmol), 5-fluoropyridin-3-ylboronic acid (33 mg, 0.24 mmol), K₂CO₃ (65 mg, 0.47 mmol) and Pd(dppf)Cl₂ (38 mg, 0.05 mmol) in 1,4-dioxane:H₂O (4.0 mL, 3:1) was stirred at 100° C. for 2 h. The reaction was cooled to rt and concentrated in vacuo. The residue was purified on silica, (pet ether/EtOAc 1:1) and prep-HPLC (C18, H₂O (0.1% formic acid):MeCN, 1:1 to 1:4) to afford the title compound (14 mg, 14%) as a white solid. LCMS (Method E) 1.85 min, [M+H]⁺=442.15, gradient 5-100% B. HRMS m/z: [M+H]⁺ calculated for C₂₂H₁₆F₄N₅O 442.1291; found 442.1292.

### Example 9

### 3-[4-(5-methoxypyridin-3-yl)-1H-pyrazol-1-yl]-4-methyl-N-[4-(trifluoromethyl)pyridin-2-yl]benzamide

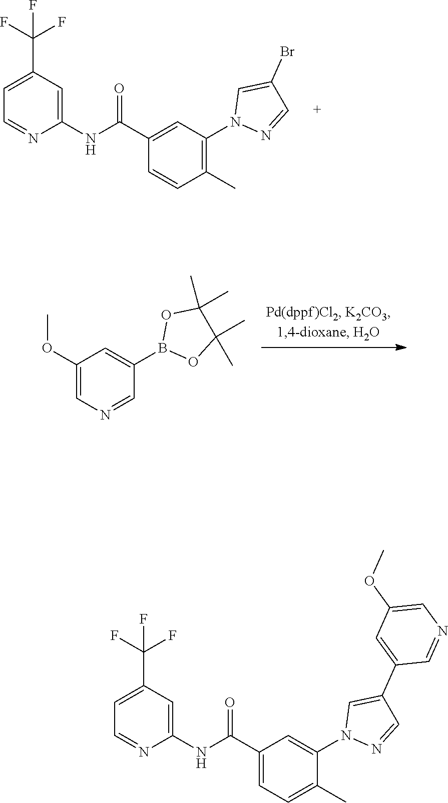

A mixture of Intermediate 5 (100 mg, 0.24 mmol), 3-methoxy-5-(4,4,5,5-tetramethyl-1,3,2-dioxaborolan-2-yl)pyridine (55 mg, 0.24 mmol), K₂CO₃ (65 mg, 0.47 mmol) and Pd(dppf)Cl₂ (38 mg, 0.05 mmol) in 1,4-dioxane:H₂O (4.0 mL, 3:1) was stirred at 100° C. for 2 h. The reaction was cooled to rt then filtered and the filter cake washed with EtOAc (50 mL). The resulting solution was concentrated in vacuo and the crude residue purified by prep-HPLC (C18, H₂O (0.1% formic acid):MeCN, 11:9 to 2:3) to afford the title compound (30 mg, 28%) as a white solid. LCMS (Method E) 1.66 min, [M+H]⁺=454.20, gradient 5-55% B. HRMS m/z: [M+H]⁺ calculated for C₂₃H₁₉F₃N₅O₂ 454.1491; found 454.1490.

### Example 10

### N-[6-cyano-4-(trifluoromethyl)pyridin-2-yl]-4-methyl-3-[4-(pyridin-3-yl)-1H-pyrazol-1-yl]benzamide

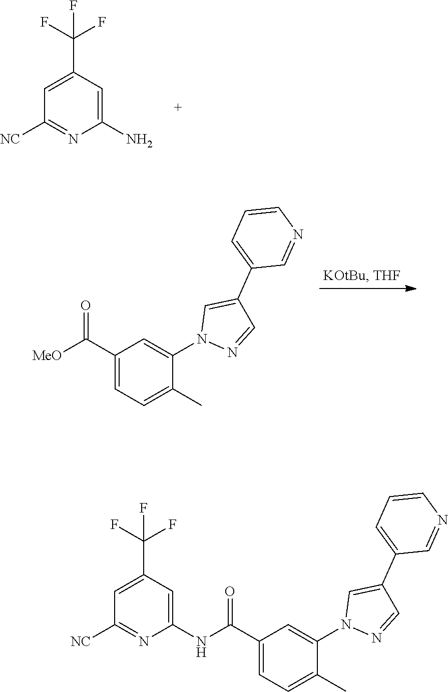 [SMILES: N#Cc1cc(C(F)(F)F)cc(N)n1 ; COC(=O)c1ccc(C)c(-n2cc(-c3cccnc3)cn2)c1 ; Cc1ccc(C(=O)Nc2cc(C(F)(F)F)cc(C#N)n2)cc1-n1cc(-c2cccnc2)cn1]

To a solution of Intermediate 3 (10 mg, 0.03 mmol) and 6-amino-4-(trifluoromethyl)pyridine-2-carbonitrile (6 mg, 0.03 mmol) in THF (1.0 mL) was added KOtBu (15 mg, 0.14 mmol) and the reaction mixture was stirred at rt for 2 h. The reaction was quenched with H₂O and the resulting mixture was extracted with EtOAc (3×5 mL). The combined organics were washed with brine (2×5 mL), dried over Na₂SO₄, filtered and concentrated in vacuo. The crude product was purified by prep-HPLC (RP18 OBD column, H₂O (10 mM NH₄HCO₃):MeCN, 11:9 to 43:57) to give the title compound (2 mg, 8%) as light yellow solid. LCMS (Method F) 1.56 min, [M+H]⁺=449.1. HRMS m/z: [M+H]⁺ calculated for C₂₃H₁₆F₃N₆O 449.1338; found 449.1339.

### Comparative Example 1

### 4-Methyl-3-[4-(3-pyridyl)pyrazol-1-yl]-N-[3-(pyrrolidin-1-ylmethyl)-5-(trifluoromethyl)phenyl]benzamide

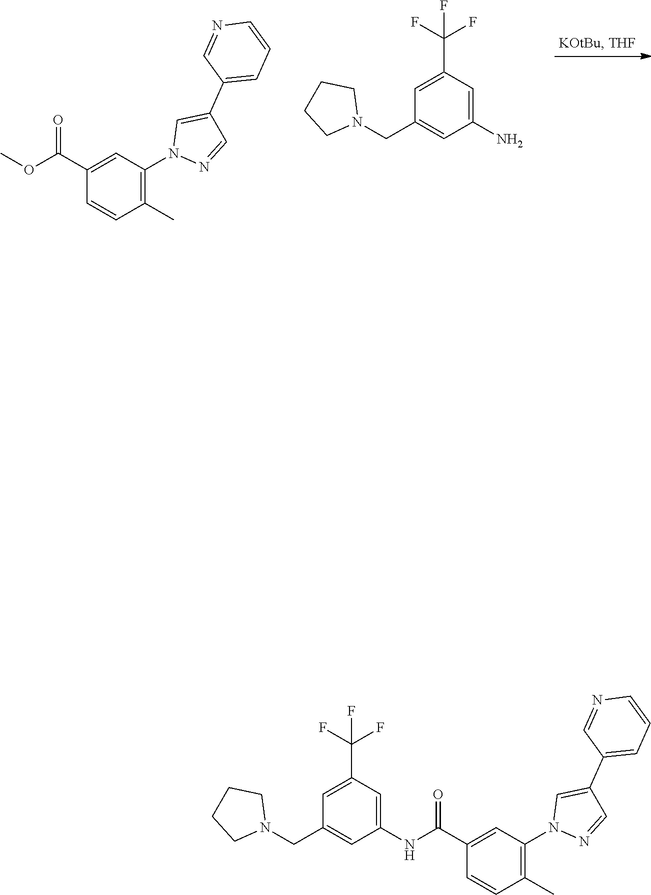 [SMILES: COC(=O)c1ccc(C)c(-n2cc(-c3cccnc3)cn2)c1 ; Nc1cc(CN2CCCC2)cc(C(F)(F)F)c1 ; Cc1ccc(C(=O)Nc2cc(CN3CCCC3)cc(C(F)(F)F)c2)cc1-n1cc(-c2cccnc2)cn1]

To a solution of Intermediate 3 (101 mg, 0.34 mmol) and Intermediate 12 (70 mg, 0.29 mmol) in THF (3.0 mL) was added KOtBu (20% sol THF, 0.84 mL, 1.43 mmol) and the reaction mixture stirred at rt for 1 h. The reaction was quenched with brine (15 mL) and extracted with EtOAc (3×15 mL). The combined organics were dried (MgSO₄) and concentrated. The resulting oil was purified on silica (Biotage Isolera, reverse phase, MeCN:H₂O, 1:9 to 4:1, both eluents containing 0.1 Vol % NH₃) to give the title compound (21 mg, 14%) as an off white solid. UPLC (Method A) 3.62 min, 97.2%, [M+H]⁺=506.3.

### Comparative Example 2

### 4-Methyl-N-[3-(morpholinomethyl)-5-(trifluoromethyl)phenyl]-3-[4-(3-pyridyl)pyrazol-1-yl]benzamide

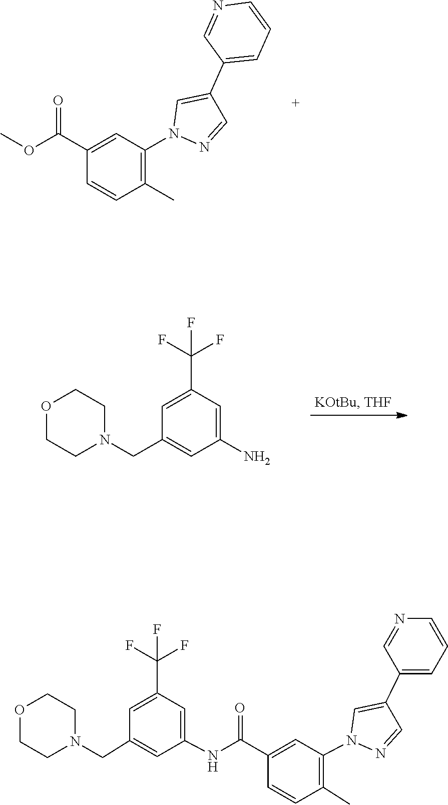 [SMILES: COC(=O)c1ccc(C)c(-n2cc(-c3cccnc3)cn2)c1 ; Nc1cc(CN2CCOCC2)cc(C(F)(F)F)c1 ; Cc1ccc(C(=O)Nc2cc(CN3CCOCC3)cc(C(F)(F)F)c2)cc1-n1cc(-c2cccnc2)cn1]

To a solution of Intermediate 3 (74.4 mg, 0.25 mmol) and Intermediate 15 (55.0 mg, 0.21 mmol) in THF (2.0 mL) was added KOtBu (20% sol THF, 0.62 mL, 1.06 mmol) and the reaction mixture was stirred at rt for 1 h. The reaction was quenched with brine (15 mL) and extracted with EtOAc (3×15 mL). The combined organics were dried over MgSO₄ and concentrated. The resulting oil was purified on silica (Biotage Isolera, reverse phase, MeCN:H₂O, 1:9 to 4:1, both eluents containing 0.1 Vol % NH₃) to give the title compound (49 mg, 44%) as an off white solid. UPLC (Method A) 3.34 min, 99.7%, [M+H]⁺=522.2.

### Comparative Example 3

### 4-Methyl-3-[4-(3-pyridyl)triazol-1-yl]-N-[5-(trifluoromethyl)pyridazin-3-yl]benzamide

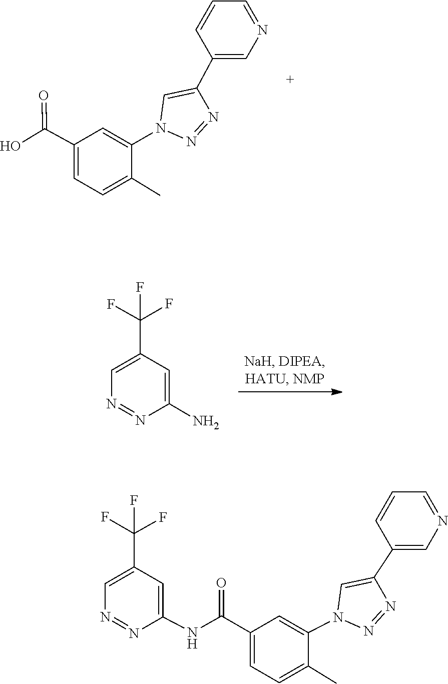 [SMILES: Cc1ccc(C(=O)O)cc1-n1cc(-c2cccnc2)nn1 ; Nc1cc(C(F)(F)F)cnn1 ; Cc1ccc(C(=O)Nc2cc(C(F)(F)F)cnn2)cc1-n1cc(-c2cccnc2)nn1]

A stirring solution of 5-(trifluoromethyl)pyridazin-3-amine (149 mg, 0.91 mmol) in anhydrous NMP (2.0 mL) under N₂ at rt was treated with NaH (60% in oil) (73 mg, 1.84 mmol) and stirred for 50 min. A stirring solution of Intermediate 18 (85 mg, 0.30 mmol) and DIPEA (0.10 mL, 0.57 mmol) in NMP (1.5 mL) at rt was treated with HATU (175 mg, 0.46 mmol) and stirred for 50 min. The DIPEA/HATU solution was then added dropwise to the NaH solution at rt and the reaction was stirred at rt for 20 h. The reaction was diluted with H₂O (10 mL) and extracted with EtOAc (4×10 mL), the combined organics were washed with brine (10 mL), dried over Na₂SO₄, filtered and concentrated to give the crude product. The residue was purified on silica (Biotage Isolera, reverse phase, MeCN:H₂O, 1:9 to 4:1, both eluents containing 0.1 Vol % NH₃) to give two batches of the title compound batch 1 (3.50 mg, 3%) and batch 2 (2.50 mg, 2%), both as beige powders. UPLC (Method C) 2.87 min, 95.6%, [M+H]⁺=426.2.

### Comparative Example 4

### 4-Methyl-3-[3-(3-pyridyl)pyrazol-1-yl]-N-[4-(trifluoromethyl)-2-pyridyl]benzamide

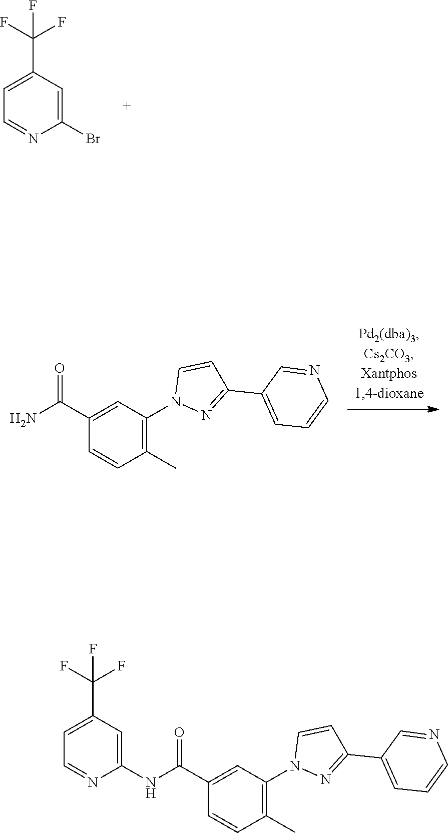 [SMILES: FC(F)(F)c1ccnc(Br)c1 ; Cc1ccc(C(N)=O)cc1-n1ccc(-c2cccnc2)n1 ; Cc1ccc(C(=O)Nc2cc(C(F)(F)F)ccn2)cc1-n1ccc(-c2cccnc2)n1]

To 2-bromo-4-(trifluoromethyl)pyridine (146 mg, 0.65 mmol), Intermediate 21 (90 mg, 0.32 mmol), Cs₂CO₃ (316 mg, 0.97 mmol) in 1,4-dioxane (1.0 mL) was added Xantphos (41 mg, 0.07 mmol) and Pd₂(dba)₃ (30 mg, 0.03 mmol) under N₂ and the mixture heated to 100° C. overnight. The volatiles were removed in vacuo and the residue was further purified on silica (Biotage Isolera, reverse phase, MeCN:H₂O, 1:9 to 4:1, both eluents containing 0.1 Vol % NH₃). The resulting residue was further purified via trituration with MeCN (5.0 mL) to provide the title compound (14.0 mg, 10%). UPLC (Method A) 3.44 min, 100%, [M+H]⁺=424.2.

### Comparative Example 5

### 3-[4-(2-aminopyrimidin-5-yl)triazol-1-yl]-4-methyl-N-[4-[(4-methylpiperazin-1-yl)methyl]-3-(trifluoromethyl)phenyl]benzamide

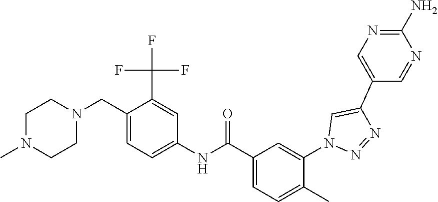 [SMILES: Cc1ccc(C(=O)Nc2ccc(CN3CCN(C)CC3)c(C(F)(F)F)c2)cc1-n1cc(-c2cnc(N)nc2)nn1]

Synthesised using the conditions described in CN103539784. UPLC (Method A) 2.96 min, 96.2%, [M+H]⁺=552.3.

### Comparative Example 6

### 4-Methyl-N-[5-[(4-methylpiperazin-1-yl)methyl]-4-(trifluoromethyl)-2-pyridyl]-3-[4-(3-pyridyl)pyrazol-1-yl]benzamide

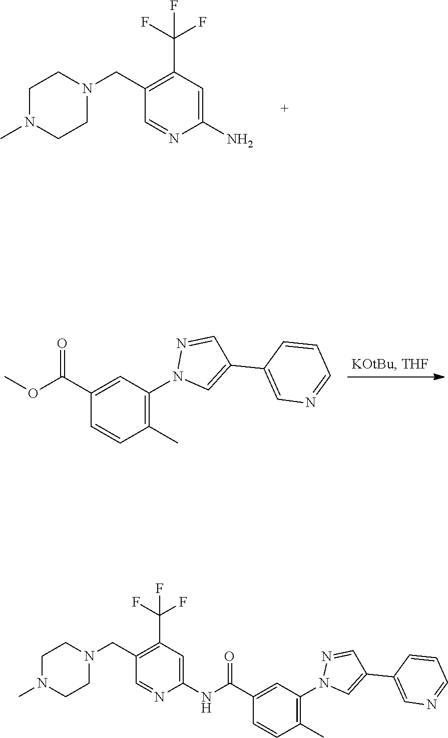 [SMILES: CN1CCN(Cc2cnc(N)cc2C(F)(F)F)CC1 ; COC(=O)c1ccc(C)c(-n2cc(-c3cccnc3)cn2)c1 ; Cc1ccc(C(=O)Nc2cc(C(F)(F)F)c(CN3CCN(C)CC3)cn2)cc1-n1cc(-c2cccnc2)cn1]

To Intermediate 3 (68 mg, 0.23 mmol) and 5-[(4-methylpiperazin-1-yl)methyl]-4-(trifluoromethyl)pyridin-2-amine (the synthesis of which is disclosed in WO2018/103751) (54 mg, 0.19 mmol) in THF (5.0 mL) at 0° C. was added KOtBu (582 μL, 0.58 mmol) under N₂. The reaction was stirred for 2 h and then warmed to rt over 16 h. The reaction was cooled to 0° C. and further Intermediate 3 (34 mg, 0.12 mmol) and KO^{t}Bu (582 μL, 0.58 mmol) were added and the reaction stirred for 1 h. After 1h further warming to rt, the reaction was quenched with water (5 mL) and extracted with EtOAc (2×5 mL). The organics were dried and concentrated to afford the crude product. The residue was purified on silica (Biotage Isolera, 0% to 10% MeOH in DCM with 1% Et₃N) and again (Biotage Isolera, 5% to 95% MeCN in H₂O, both eluents containing 0.1 Vol % NH₃) to yield the title compound (16 mg, 15%) as a beige solid. UPLC (Method A) 3.30 min, 98.1%, [M+H]⁺=536.2.

### HRMS Data

| Example | 1 | 2 | 3 | 4 | 5 | 6 |
|---|---|---|---|---|---|---|
| MWt | 423.39 | 424.38 | 424.38 | 440.42 | 453.42 | 521.54 |
| MIM | 423.13071 | 424.12595 | 424.12595 | 440.15726 | 453.15250 | 521.21509 |
| Positive | 424.1355 | 425.1308 | 425.1307 | 441.1621 | 454.1574 | 522.2200 |
| ion m/z | | | | | | |
| Negative | 422.1224 | 423.1175 | 423.1178 | 439.1488 | 452.1439 | 520.2064 |
| ion m/z | | | | | | |
| Measured | 423.1282 | 424.1235 | 424.1234 | 440.1548 | 453.1501 | 521.2127 |
| MIM from | | | | | | |
| positive | | | | | | |
| ion* | | | | | | |
| Measured | 423.1297 | 424.1248 | 424.1251 | 440.1561 | 453.1512 | 521.2137 |
| MIM from | | | | | | |
| negative | | | | | | |
| ion* | | | | | | |

*Calculated MIM assumes detection of protonated and deprotonated species in positive and negative ion mode respectively.

### BIOLOGICAL DATA

### Ba/F3 CellTiter-Glo Assay

The CellTiter-Glo luminescent cell viability assay is a homogeneous method of determining the number of viable cells in culture based on quantification of the ATP present. Briefly, IL-3 dependent Ba/F3 cells are modified to express BCR-ABL. Activity of the transformed kinase overrides IL-3 dependency for cellular proliferation and survival. Test compounds that specifically inhibit kinase activity lead to programmed cell death which can be measured through the addition of CellTiter-Glo reagent. In this assay Ba/F3 cells expressing BCR-ABL (Advanced Cellular Dynamics) or parental Ba/F3 (control) cells were prepared at 5×10⁴/mL in RPMI 1640 containing 10% FBS, 1×Glutamax and 750 ng/mL puromycin. Test compounds were dispensed into 384 well plates using the Tecan D300e at a top final assay concentration of 10 μM with dosing normalised to 0.1% DMSO in 50 μL volume. 50 μL cells were added to each well of the prepared 384 well plates and the plates spun at 1000 rpm for 1 min prior to incubation at 37° C., 5% CO₂ for 48 h. After 48 h 15 μL CellTiter-Glo reagent was added to each well in the plate. Following a 60 min incubation at rt luminescence was read on the Pherastar FS reader.

The exemplified compounds of the invention were tested in the Ba/F3 CellTiter-Glo Assay and the pIC₅₀ data is shown in the table below. pIC₅₀ data are calculated as the −log₁₀(IC₅₀ in Molar). Those data show that the compounds of the invention can inhibit c-Abl. The data demonstrates the importance of the regioisomer of the pyrazole in the core of Formula (I). Moving group Y from the 4-position on the pyrazole (Example 1) to the 3-position (Comparative Example 4) substantially decreases inhibition (24 nM vs 2710 nM).

| Example | IC₅₀ (nM) |
|---|---|
| 1 | 22 |
| 3 | 25 |
| 2 | 40 |
| 4 | 72 |
| 5 | 54 |
| 6 | 83 |
| 7 | 32 |
| 8 | 29 |
| 9 | 28 |
| 10 | 18 |
| Comparative example 4 | 2710 |

### MDR1 and BCRP-MDCK: Effective Efflux Ratio

Wild-type MDCK, MDR1-MDCK and BCRP-MDCK cells were seeded into 24 well Transwell plates and cultured for 3 days to form monolayers. Test compound were prepared at 1 μM in Hanks' Balanced Salt Solution containing 25 mM HEPES and loaded into the donor compartments of Transwell plates bearing the cell monolayers (pH 7.4 for both donor and receiver compartments). Lucifer Yellow was added to the apical buffer in all wells to assess integrity of the cell monolayer. Duplicate wells were prepared and incubated at 37° C. in a CO₂ incubator. Samples were removed at time zero and 60 minutes and test compound analysed by LC-MS/MS. Concentrations of Lucifer Yellow in the samples was measured using a fluorescence plate reader. The apparent permeability (Papp) values of test compound was determined for both the apical to basal (A>B) and basal to apical (B>A) permeation and the efflux ratio (B>A: A>B) determined in each cell line. The effective efflux ratio was determined from the ratio of either MDR1-MDCK cells or BCRP-MDCK cells relative to the ratio observed in wild-type cells. A higher value represent a better substrate for the efflux mechanism, which is less preferable. These data show that the compounds of the invention are poor substrates for the efflux mechanisms.

| Example | MDCK MDR1 EER | MDCK BCRP EER |
|---|---|---|
| 1 | 1 | 1.5 |
| 2 | 2.9 | 1.5 |
| 3 | 1.0 | 1.0 |
| 4 | 1.2 | 1.3 |
| 5 | 1.1 | 1.0 |
| 7 | 1.2 | 0.8 |
| 8 | 1.6 | 1.1 |
| 9 | 1.2 | 1.1 |
| Comparative | 6.5 | 1.8 |
| example 2 | | |
| Comparative | 9.8 | 8.5 |
| example 3 | | |
| Comparative | 4.3 | 2.1 |
| example 5 | | |
| Comparative | 15.8 | 5.0 |
| example 6 | | |

### Human/Rat Microsomal Stability

This assay measures the metabolic stability of a given compound and predicts intrinsic liver clearance for various species. A quench plate is initially prepared which contains 400 μL MeCN and 25 ng/mL of internal standard. An aliquot of microsomes is defrosted and subsequently diluted with buffer. A solution of NADPH is made up fresh just before initialising the assay. 445 μL of microsome solution is added to the desired compound (5 μL in 10 μM DMSO) and the resulting solution mixed. The reaction is initiated by the addition of 50 μL of NADPH, a T0 sample is removed before the addition and quenched. The sample plate is kept at a constant 37° C. during the incubation. At pre-determined timepoints (2.5, 5, 10, 20 and 40 min) the incubation is mixed and 22.5 μL samples are transferred to quench plates. After completion of the time course, the quench plates are sealed and shaken for a minimum of 5 min, before centrifugation at 4000 rpm for 5 min. The supernatant is then subsequently transferred to a fresh plate and diluted 1:3 with MeOH/H₂O. The plates are then heat sealed and analysed by a Water Acquity UPLC and a Waters TQ-S mass spectrometer. Waters MassLynx software is used for analysis using an MRM transition that has been previously generated from prior optimisation. After acquiring the data on the system the chromatograms are processed and checked using TargetLynx XS software and the raw data output is copied into a microsomal stability workbook. The workbook automatically populates and derives the CLᵢₙₜ, t_{1/2} and scales the CLᵢₙₜ values to generate Eh and predicted CL values. The analyst has the final workbook quality checked before the experiment is signed off. The data shows that compounds of the invention are more stable than comparative compounds.

| | Microsomal stability |
|---|---|
| Example | (human/rat) Eh |
| 1 | 0.70/0.69 |
| 2 | 0.56/0.67 |
| 3 | 0.56/0.44 |
| 4 | <0.44/0.60 |
| 10 | 0.70/0.65 |
| Comparative example 1 | 0.81/0.93 |
| Comparative example 2 | 0.96/0.99 |

The data above show that compounds of the invention have a better mosaic of properties, particularly in terms of their efficacy, microsomal stability, and resistance to efflux, than comparative compounds. This means that they are particular suitable for use in the range of treatment described above.

1. A compound of Formula (I) 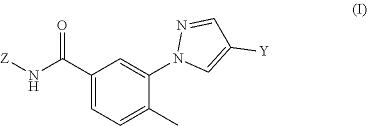
or a pharmaceutically acceptable salt, solvate, hydrate, tautomer, optical isomer, N-oxide, and/or prodrug thereof, wherein
Y is 5- or 6-membered heteroaryl optionally substituted with one or more substituents independently selected from the group consisting of:
(i) C₁-C₆ alkyl, C₂-C₆ alkenyl, C₂-C₆ alkynyl, and C₁-C₆ alkoxy, each of which is optionally substituted with one or more substituents independently selected from —NR¹R², —OR³, halo, and oxo;
(ii) halo, nitro, —CN, —C(O)NR⁴R⁵, —NR⁴R⁵, —C(O)OR⁶, —C(O)R⁶, and —OH; and
(iii) C₆-C₁₀ aryl, C₁-C₉ heteroaryl, and C₁-C₉ heterocycle, each of which is optionally substituted with one or more substituents independently selected from halo, C₁-C₆ alkyl, and C₁-C₆ haloalkyl;
Z is a 5- or 6-membered heteroaryl optionally substituted with one or more substituents selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR⁷R⁸, —OR⁹, halo, and oxo; and
(ii) halo, nitro, —CN, —C(O)NR¹⁰R¹¹, —NR¹⁰R¹¹, —C(O)OR¹², —C(O)R¹², and —OH; and
each of R¹ to R¹² is independently selected from the group consisting of H, C₁-C₆ alkyl, and C₁-C₆ haloalkyl; or
R¹ and R² and/or R⁴ and R⁵ may be taken together with the nitrogen atom to which they are respectively attached to form a 4- to 6-membered monocyclic heterocyclic ring that is optionally substituted with one or more substituents independently selected from halo, C₁-C₆ alkyl, and C₁-C₆ haloalkyl.

2. The compound according to claim 1, wherein Y is selected from the group consisting of optionally substituted pyrrolyl, pyrazolyl, imidazolyl, triazolyl, tetrazolyl, oxazolyl, isoxazolyl, thiazolyl, isothiazolyl, oxadiazolyl, thiadiazolyl, pyridyl, pyridazinyl, pyrimidinyl, pyrazinyl, and triazinyl,
wherein the optionally substitution is with one or more substituents independently selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR¹R², —OR³, halo, and oxo;
(ii) halo, nitro, —CN, —C(O)NR⁴R⁵, —NR⁴R⁵, —C(O)OR⁶, —C(O)R⁶, and —OH; and
(iii) C₆-C₁₀ aryl, C₁-C₉ heteroaryl, and C₁-C₉ heterocycle, each of which is optionally substituted with one or more substituents independently selected from halo, C₁-C₆ alkyl, and C₁-C₆ haloalkyl.

3. The compound according to claim 1, wherein Y is selected from one of the following groups 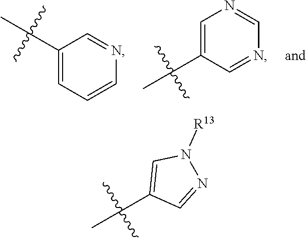
each of which being optionally substituted with one or more substituents independently selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR¹R², —OR³, halo, and oxo;
(ii) halo, nitro, —CN, —C(O)NR⁴R⁵, —NR⁴R⁵, —C(O)OR⁶, —C(O)R⁶, and —OH; and
(iii) C₆-C₁₀ aryl, C₁-C₉ heteroaryl, and C₁-C₉ heterocycle, each of which is optionally substituted with one or more substituents independently selected from halo, C₁-C₆ alkyl, and C₁-C₆ haloalkyl; and
wherein R¹³ is selected from the group selected from the group consisting of H, C₁-C₆ alkyl and C₁-C₆ haloalkyl.

4. The compound according to claim 1, wherein Y is optionally substituted with one or more substituents independently selected from the group consisting of
(i) C₁-C₆ alkyl, and C₁-C₆ alkoxy, each of which is optionally substituted with one or more substituents independently selected from —NR¹R², —OR³, halo, and oxo; and
(ii) halo, —C(O)NR⁴R⁵, —NR⁴R⁵, —C(O)OR⁶, —C(O)R⁶, and —OH.

5. The compound according to claim 1, wherein Y is optionally substituted with one or more substituents independently selected from the group consisting of —OH, halo, C₁-C₆ alkyl, C₁-C₆ alkoxy, C₁-C₆ haloalkyl, and —NR⁴R⁵, wherein R⁴ and R⁵ are independently selected from the group consisting of H, C₁-C₃ alkyl, and C₁-C₃ haloalkyl.

6. The compound according to claim 1, wherein Y is optionally substituted with one or more substituents independently selected from the group consisting of —OH, C₁-C₆ alkyl, C₁-C₆ alkoxy, C₁-C₆ haloalkyl, and —NR⁴R⁵, wherein R⁴ and R⁵ are independently selected from the group consisting of H, C₁-C₃ alkyl, and C₁-C₃ haloalkyl.

7. The compound according to claim 1, wherein Z is selected from the group consisting of pyrrolyl, pyrazolyl, imidazolyl, triazolyl, tetrazolyl, oxazolyl, isoxazolyl, thiazolyl, isothiazolyl, oxadiazolyl, thiadiazolyl, pyridyl, pyridazinyl, pyrimidinyl, pyrazinyl, and triazinyl,
wherein the optionally substitution is with one or more substituents selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR⁷R⁸, —OR⁹, halo, and oxo; and
(ii) halo, nitro, —CN, —C(O)NR¹⁰R¹¹, —NR¹⁰R¹¹, —C(O)OR¹², —C(O)R¹², and —OH.

8. The compound according to claim 1, wherein Z is selected from one of the following groups 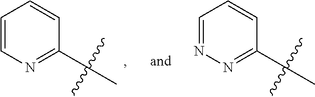 [SMILES: *c1ccccn1 ; *c1cccnn1]
each of which is optionally substituted with one or more substituents selected from the group consisting of
(i) C₁-C₆ alkyl, C₂-C₆ alkenyl, C₂-C₆ alkynyl, and C₁-C₆ alkoxy, each of which is optionally substituted with one or more substituents independently selected from —NR⁷R⁸, —OR⁹, halo, and oxo; and
(ii) halo, nitro, —CN, —C(O)NR¹⁰R¹¹, —NR¹⁰R¹¹, —C(O)OR¹², —C(O)R¹², and —OH.

9. The compound according to claim 1, wherein Z is optionally substituted with one or more substituents independently selected from the group consisting of
(i) C₁-C₆ alkyl and C₁-C₆ alkoxy, each of which is optionally substituted with one or more substituents independently selected from —NR¹R², —OR³, halo, and oxo; and
(ii) halo, nitro, —CN, —C(O)NR¹⁰R¹¹, —NR¹⁰R¹¹, —C(O)OR¹², —C(O)R¹², and —OH.

10. The compound according to claim 1, wherein Z is optionally substituted with one or more substituents independently selected from the group consisting of halo, —CN, and C₁-C₆ haloalkyl.

11. The compound according to claim 1, wherein Z is optionally substituted with one or more substituents independently selected from the group consisting of halo and C₁-C₆ haloalkyl.

12. The compound according to claim 1, wherein
Y is optionally substituted with one or more substituents independently selected from the group consisting of —OH, halo, C₁-C₃ alkyl, C₁-C₃ alkoxy, C₁-C₃ haloalkyl, and —NR⁴R⁵;
R⁴ and R⁵ are independently selected from the group consisting of H, C₁-C₃ alkyl, and C₁-C₃ haloalkyl; or
R⁴ and R⁵ may be taken together with the nitrogen atom to which they are respectively attached to form a 5- to 6-membered monocyclic heterocyclic ring that is optionally substituted with one or more substituents independently selected from halo, C₁-C₃ alkyl, and C₁-C₃ haloalkyl;
Z is selected from one of the following groups 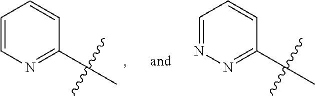 [SMILES: *c1ccccn1 ; *c1cccnn1]
each of which is optionally substituted with one or more substituents independently selected from the group consisting of halo, —CN, and C₁-C₃ haloalkyl.

13. The compound according to claim 1, wherein
Y is optionally substituted with one or more substituents independently selected from the group consisting of —OH, C₁-C₃ alkyl, C₁-C₃ alkoxy, C₁-C₃ haloalkyl, and —NR⁴R⁵;
R⁴ and R⁵ are independently selected from the group consisting of H, C₁-C₃ alkyl, and C₁-C₃ haloalkyl; or
R⁴ and R⁵ may be taken together with the nitrogen atom to which they are respectively attached to form a 5- to 6-membered monocyclic heterocyclic ring that is optionally substituted with one or more substituents independently selected from halo, C₁-C₃ alkyl, and C₁-C₃ haloalkyl;
Z is selected from one of the following groups 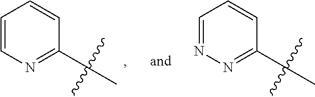 [SMILES: *c1ccccn1 ; *c1cccnn1]
each of which is optionally substituted with one or more substituents independently selected from the group consisting of halo and C₁-C₃ haloalkyl.

14. The compound according to claim 12, wherein Y is substituted with a substituent selected from the group consisting of —NH(C₁-C₃)alkyl and 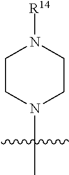
wherein R¹⁴ is selected from the group consisting of H, C₁-C₃ alkyl and C₁-C₃ haloalkyl.

15. The compound according to claim 1, wherein the compound of Formula (I) is selected from the group consisting of
4-Methyl-3-[4-(3-pyridyl)pyrazol-1-yl]-N-[4-(trifluoromethyl)-2-pyridyl]benzamide;
4-Methyl-3-(4-pyrimidin-5-ylpyrazol-1-yl)-N-[4-(trifluoromethyl)-2-pyridyl]benzamide;
4-Methyl-3-[4-(3-pyridyl)pyrazol-1-yl]-N-[5-(trifluoromethyl)pyridazin-3-yl]benzamide;
3-[4-(1,5-Dimethylpyrazol-4-yl)pyrazol-1-yl]-4-methyl-N-[4-(trifluoromethyl)-2-pyridyl]benzamide;
4-Methyl-3-[4-[2-(methylamino)pyrimidin-5-yl]pyrazol-1-yl]-N-[4-(trifluoromethyl)-2-pyridyl]benzamide;
4-Methyl-3-[4-[5-(4-methylpiperazin-1-yl)-3-pyridyl]pyrazol-1-yl]-N-[4-(trifluoromethyl)-2-pyridyl]benzamide;
4-Methyl-3-[4-(5-methylpyridin-3-yl)-1H-pyrazol-1-yl]-N-[4-(trifluoromethyl)pyridin-2-yl]benzamide;
3-[4-(5-Fluoropyridin-3-yl)-1H-pyrazol-1-yl]-4-methyl-N-[4-(trifluoromethyl)pyridin-2-yl]benzamide;
3-[4-(5-methoxypyridin-3-yl)-1H-pyrazol-1-yl]-4-methyl-N-[4-(trifluoromethyl)pyridin-2-yl]benzamide; and
N-[6-cyano-4-(trifluoromethyl)pyridin-2-yl]-4-methyl-3-[4-(pyridin-3-yl)-1H-pyrazol-1-yl]benzamide,
or a pharmaceutically acceptable salt, solvate, hydrate, tautomer, optical isomer, N-oxide, and/or prodrug thereof.

16. A pharmaceutical composition comprising a compound according to claim 1 and a pharmaceutically acceptable carrier, excipient, and/or diluent.

17.-19. (canceled)

20. A method for the treatment or prevention of a disease or condition responsive to c-ABL inhibition comprising administering a therapeutically effective amount of the compound according to claim 1 to a subject in need thereof.

21. The method of claim 20, wherein the disease or condition is a neurodegenerative disorder, a cancer, a prion disease, a viral infection, diabetes, an inflammatory disease, or a skeletal or muscular dystrophy.

22. The method of claim 20, wherein the disease or condition is a neurodegenerative disorder and the neurodegenerative disorder is selected from the group consisting of Alzheimer disease, Down's syndrome, frontotemporal dementia, progressive supranuclear palsy, Pick's disease, Niemann-Pick disease, Parkinson's disease, Huntington's disease (HD), dentatorubropallidoluysian atrophy, Kennedy's disease, spinocerebellar ataxia, fragile X (Rett's) syndrome, fragile XE mental retardation, Friedreich's ataxia, myotonic dystrophy, spinocerebellar ataxia type 8, spinocerebellar ataxia type 12, Alexander disease, Alper's disease, amyotrophic lateral sclerosis (ALS), ataxia telangiectasia, Batten disease, Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

23. The method of claim 22, wherein the neurodegenerative disorder is amyotrophic lateral sclerosis (ALS) or Parkinson's disease.

24. The method of claim 20, wherein the disease or condition is cancer and the cancer is leukaemia, acute lymphoblastic leukaemia (ALL), acute myelogenous leukaemia (AML), or mixed-phenotype acute leukaemia (MPAL), or any central nervous system (CNS) metastases thereof.
